(19) United States
(12) Patent Application Publication
(10) Pub. No.: US 2005/0220906 A1
(43) Pub. Date: Oct. 6, 2005

(54) Pharmaceutical composition for treating rheumatism and the preparation thereof

(76) Inventor: Wenlong Deng, Chengdu City (CN)

Correspondence Address: CHRISTIE, PARKER & HALE, LLP, PO BOX 7068, PASADENA, CA, 91109-7068, US

(21) Appl. No.: 10/510,617

(22) PCT Filed: Apr. 9, 2002
(86) PCT No.: PCT/CN02/00246

§ 371 Date: Apr. 25, 2005

(51) Int. Cl. A61K 35/78

(52) U.S. Cl. 424/759; 424/761; 424/769

(57) ABSTRACT

The invention has brought to light a kind of antirheumatic and its preparation, which was made from Tripterygium hypoglaucum (Levl.) Hutch, Epimedium brevicornum Maxim, Lycium barbarum L, and Cuscuta chinensis Lam. The invented medicine has the merits of prominent effect, mild side reaction and convenient administration.

### THE FIELD OF THE INVENTION

The invention is about a medicine which is used to treat rheumatism and its preparation.

### THE BACKGROUND OF THE INVENTION

It is believed that the rheumatoid arthritis (RA) is refractory and about 18,000,000 RA patients have been disabled because of this disease. The medicine reseach for curing RA has continued about a century. Aspirin is the first medicine which is widely used to treat RA. The medicine to treat RA can be divided into 2 kinds: non-steroidal anti-inflammatory drugs (NSAIDs) and immunosuppressive agent. NSAIDs includes cyclophthasine, antinfan and adrenal cortex hormone. The clinical researchs have proved the effectiveness of NSAIDs. The immuneosuppressive agent includes methotrexate, cyclophosphane, penicillamine and et al. The immunoregulation has become one of the important theropies in the recent years. But all the medicines which are used to treat rheumatism have serious side-effect. The medicine which can treat rheumatism effectively and non-poisonously hasn't invented by now.

There are 3 directions in the research of antirheumatic should be emphasized. The first direction is NSAIDs and cytokine-antagon, such as recombined soluble TNF α ntagon, IL-1 inhibitor and PAF inhibitor. The second direction is the new immunosuppressive agent and immunomodulator, such as cyclosporin A. The third direction is the compound medicines.

In the TCM, the research on the “arthralgia disease” (equals to the definition of rheumatism in the modern medicine) can be traced back to the Han dynasty more than 1,500 years ago. Three prescriptions: “Ma Xing Shi Gan decoction”, “Fangji Hangqi decoction” and “Wutong decoction”, which is used to treat “Bi Zheng” were recorded in the medicine classics “Shanghan Lun” wroted by the famous doctor Zhang Zhongjing at that time. Gelsemium elegan s Bentll is a kind of wild plant in Sichuang province and it has been proved effective to treat rheumatism in a clinical research carried at the local area. But the further study found that it had a serious side-effect on the reproduction organs and some other uncontrollable problem.

The treatment of “arthralgia disease” by the method of TCM has reached a high level by numerous doctors' development in so long a history. By now, there are many effective prescriptions and herbs. There are more than 80 kinds of herbs and 29 kinds of patent medicines recorded in China pharmacopoeia 1995 edition and 2000 edition. But there are still many problems for example: \mathring{1} the effect is not good enough in treating the serious arthralgia disease such as rheumatoid arthritis; \mathring{2} the dosage forms are not fit for the modern life. \mathring{3} some medicine has good effect, but the side-effect is serious too, such as the extract of triperygium wilfordii. So that it is necessary to develop the highly-effective-lowly-noxious and convenient for administration antirheumatic medicine. This medicine should have the similar effect and the lower side effect to the artificial antirheumatic medicine.

### THE CONTENT OF THE INVENTION

The invention is to supply an antirheumatic, which is highly-effective-lowly-noxious and convenient for administration, and its preparation thereof.

The invented medicne's technical proposal is realized by using the crude herbs as following:
Tripterygium hypoglaucum (Levl.) Hutch.
Epimedium brevicornum Maxim.
Lycium barbarum L.
Cuscuta chinensis Lam. (or Cuscuta australis R. Br.)

The invented medicine is made from the crude herbs above.

The material to produce the invented medicine can be combined on several ways. The tripterygium hypoglaucum (Levl.) Hutch. is the necessary herb, one or two or three of the other three herbs can be added to made the material.

One of the optimal crude herbs rate of the material is as following:

| | |
|---|---|
| Tripterygium hypoglaucum (Levl.) Hutch. | 1-4 weightinweight |
| Epimedium brevicornum Maxim. | 1-4 weightinweight |
| Lycium barbarum L. | 1-4 weightinweight |
| Cuscuta chinensis Lam. | 1-4 weightinweight |

The other optimal crude herbs rate of the material is as following:

| | |
|---|---|
| Tripterygium hypoglaucum (Levl.) Hutch. | 2 weightinweight |
| Epimedium brevicornum Maxim. | 2 weightinweight |
| Lycium barbarum L. | 1 weightinweight |
| Cuscuta chinensis Lam. | 1 weightinweight |

The third optimal crude herbs rate of the material is as following:

| | |
|---|---|
| Tripterygium hypoglaucum (Levl.) Hutch. | 1-4 weightinweight |
| Epimedium brevicornum Maxim. | 1-4 weightinweight |

The fourth optimal crude herbs rate of the material is as following:

| | |
|---|---|
| Tripterygium hypoglaucum (Levl.) Hutch. | 2 weightinweight |
| Epimedium brevicornum Maxim. | 2 weightinweight |

The fifth optimal crude herbs rate of the material is as following:

| | |
|---|---|
| Tripterygium hypoglaucum (Levl.) Hutch. | 1-4 weightinweight |
| Epimedium brevicornum Maxim. | 1-4 weightinweight |
| Lycium barbarum L. | 1-4 weightinweight |

The sixth optimal crude herbs rate of the material is as following:

| | |
|---|---|
| Tripterygium hypoglaucum (Levl.) Hutch. | 2 weightinweight |
| Epimedium brevicornum Maxim. | 2 weightinweight |
| Lycium barbarum L. | 1 weightinweight |

The seventh optimal crude herbs rate of the material is as following:

| | |
|---|---|
| Tripterygium hypoglaucum (Levl.) Hutch. | 1-4 weightinweight |
| Epimedium brevicornum Maxim. | 1-4 weightinweight |
| Cuscuta chinensis Lam. | 1-4 weightinweight |

The eighth optimal crude herbs rate of the material is as following:

| | |
|---|---|
| Tripterygium hypoglaucum (Levl.) Hutch. | 2 weightinweight |
| Epimedium brevicornum Maxim. | 2 weightinweight |
| Cuscuta chinensis Lam. | 1 weightinweight |

The content of the icariine (C₃₃H₄₀O₁₅) in the medicine combinations above can not be less than 2.0 mg.

The optimal crude herbs rate of the material can be the other way as following:

| | |
|---|---|
| Tripterygium hypoglaucum (Levl.) Hutch. | 1-4 weightinweight |
| Lycium barbarum L. | 1-4 weightinweight |
| And/or Cuscuta chinensis Lam. | 1-4 weightinweight |

The optimal crude herbs rate of the material can be another way as following:

| | |
|---|---|
| Tripterygium hypoglaucum (Levl.) Hutch. | 2 weightinweight |
| Lycium barbarum L. | 1 weightinweight |
| And/or Cuscuta chinensis Lam. | 1 weightinweight |

The crude herbs are prepared on the rate and then they can be made into any dosage forms used in the clinic, such as the bolus form, the powder forms, the ointment forms, the tablet forms, the sofe or hard capsule forms, the granule forms, the injection forms and so on.

The preparation method of the invented medicine is as following:

The crude herbs are prepared on the weight rate:

| | |
|---|---|
| Tripterygium hypoglaucum (Levl.) Hutch | 1-4 weightinweight |
| Epimedium brevicornum Maxim | 1-4 weightinweight |
| Lycium barbarum L | 1-4 weightinweight |
| Cuscuta chinensis Lam | 1-4 weightinweight |

The Tripterygium hypoglaucum (Levl.) Hutch. and Epimedium brevicornum Maxim are smashed. Then the powders are decocted by water for 2˜4 times separately. The Lycium barbarum L and Cuscuta chinensis Lam are soaked in the hot water (80˜95° C.) for 1˜3 times separately. The decocted fluid and the immersion fluid of the herbs are collected and added to the correspondent macroscopicvoid adsorbent resins column separately. After the adsorption, the columns are washed until the flushing liquor tunns clear. Then the columns are eluted by 60%-80% alcohol. The eluting liquors are collected from its color turning deep till the color turning very weak. Then the alcohol in the upper part of the column is pushed out by high pressure water and mixed with the eluting liquor. The mixed eluting liquor is 3˜8 times heavy of the correspondent crude herb. All the 4 eluting liquors are recycled and condensed to the specific density 1.10 separately. The condensed liquors are dehydrated by spry drying to get the extract of the crude herbs. The 4 kind of extracts are mixed uniformly to be made into any dosage forms that needed by the clinic.

The optimal preparation method of the invented medicine is as following:

The crude herbs are prepared on the weight rate:

| | |
|---|---|
| Tripterygium hypoglaucum (Levl.) Hutch | 2 weightinweight |
| Epimedium brevicornum Maxim | 2 weightinweight |
| Lycium barbarum L | 1 weightinweight |
| Cuscuta chinensis Lam | 1 weightinweight |

The Tripterygium hypoglaucum (Levl.) Hutch. Is smashed. Then the powder is added with 13, 10, 10 folds weight of the water to decoct for 3 times. Each time is 1 hour. The Epimedium brevicornum Maxim is cut to piece. Then the herb pieces is added with 15, 10, 10 folds weight of the water to decoct for 3 times. Each time is 1 hour. The Lycium barbarum L is smashed to crude powder and soaked in the hot water (80° C., 20 fold weight of the crude herb) for 3 times. Each time is 1 hour. The Cuscuta chinensis Lam is smashed to crude powder and soaked in the hot water (80° C., 31 fold weight of the crude herb) for 3 times. Each time is 1 hour. The decocted fluid and the immersion fluid of the herbs are filtrated separately and added to the correspondent macroscopicvoid adsorbent resins column JD-1 (WLD). After the adsorption, the columns are eluted by 70% alcohol. The eluting liquors are collected from its color turning deep till the color turning very weak. The alcohol is recycled from the eluting liquor. Then the rest liquor is condensed and dehydrated to get the extract powder. The 4 kind of extract powders are mixed uniformly to be made into any dosage forms that needed by the clinic.

The invented medicine can be prepared on the method as following:

The crude herbs are prepared on the weight rate recorded before. The Tripterygium hypoglaucum (Levl.) Hutch. and Epimedium brevicornum Maxim are cut into pieces. The Lycium barbarum L and Cuscuta chinensis Lam are crushed or not. The 4 kind of herbs are extracted in the 0˜95% alcohol at 10˜98° C. for 1˜4 times separately or together. The extracted liquors are mixed or not. Then the extracted liquors are condensed, dehydrated, smashed and mixed uniformly. The mixed powder can be made into any dosage form needed in the clinic.

The invented medicine can be made from the effective constituents of the 4 herbs.

The effective constituents of Epimedium brevicornum Maxim are icariine, icariside I, icariside II, and Icariin A. The effective constituents of Tripterygium hypoglaucum (Levl.) Hutch. Are diterpenes, triterpenes and alkaloids compound. The effective constituents of Lycium barbarum L and Cuscuta chinensis Lam are both flavone.

So that the crude herb Epimedium brevicornum Maxim can be replaced by one or more kinds of the effective constituents of itself, such as icariine, icariside I, icariside II, and Icariin A. The crude herb Tripterygium hypoglaucum (Levl.) Hutch. Can be replaced by one or more kinds of the effective constituents of itself, such as diterpenes, triterpenes and alkaloids compound. While the Lycium barbarum L and Cuscuta chinensis Lam can be replaced by flavone.

It has been proved by the pharmacodynamics research that the invented medicine (Fengshiping Capsule) could inhibit the primary and secondary injury adjuvant arthritis (AA). It could inhibit the delayed hypersensitivity (DTH) in the ear of the mouse caused by the 2,4dinitrofluorobenzene (DNFB). It could inhibit the antibody produce of the hemolysin and the activity of the IL-1, IL-2, IL-6 and TNF in the macrophage and splenocyte. The Fengshiping Capsule could inhibit the lymphocyte transformation induced by the ConA. It could inhibit the CD₄, CD₈ cells remarkably, especially CD₄ cells, but the rate of CD₄/CD₈ was not affected very much. There was a remarkable linear relationship between the dosage and the effect. 12˜18 g (crude medicine)/kg was the minimum effective dose. The invented medicine could inhibit the activity of the NK cells. In the effective dose, Fengshiping Capsule did not cause the atrophy of the important immune organs such as thymus and spleen, and did not inhibit the phagocytic activity of the macrophage.

The invented medicine had a remarkable antiinfalmmatory action. It could inhibit the over penetrating condition of the capillary in the mouse's abdominal cavity caused by the ethanoic acid injected. It could improve the swelling in the ear of the mouse caused by the croton oil. It could inhibit the pleuritis in the mouse and the assembling of the WBC to the CMC cyst in the rat induced by the carrageenan. But the invented medicine couldn't inhibit the rat's foot swelling induced by the carrageenan and the granuloma caused by the tampon obviously. The Fengshipng Capsule could inhibit the body-twist reaction caused by the ethanoic acid in the mouse remarkably.

### EXPERIMENTAL EXAMPLE 1

### The Effect on the Adjuvant Arthritis (AA)

1.1 The Preventing Effect on the AA of the Invented Medicine

72 isogenous SD rats of the same batch, half male and half female, 180˜220 g weight, were divided randomly into 6 groups. Each group has 12 rats. Every 6 rats lived in a cage. The perimeter of the double anklejoints and the feet of the rat were measured accurately and recorded as the normal value. All the rats were drenched by the same volume of the invented medicine on the correspondent concentration or the solution of the Xihuangqi by the gastic injection. 1 hour later, all the rats were injected with 0.1 ml Freund's complete adjuvant (FCA) under the skin of the left postpedes. In the next 30 days, all the rats were drenched with the correspondent medicine once a day on the same dosage. And in these days, the rats were measured of the perimeters of the double anklejoints and the feet once a day. In this experiment, the swelling degree (Δcm) equaled to the difference value of the perimeters measured after the FCA injection and before the FCA injection. (See the result in table 1.1 and 1.2) At the end of the experiment, the major organs of the rats were weighted. (See the table 1.3, 1.4)

TABLE 1.1
The effect of the Fengshiping on the swelling degree of the left anklejoint and foot
after the injection of FCA in the rat AA model (\overline{X} ± S)

| | Dose | Swelling degree (Δcm) | | | | | | |
|---|---|---|---|---|---|---|---|---|
| Group | (g/kg) | 1 d | 2 d | 3 d | 9 d | 12 d | 14 d | 16 d |
| Control | — | 0.69 ± 0.17 | 0.69 ± 0.12 | 0.92 ± 0.18 | 0.84 ± 0.41 | 1.10 ± 0.30 | 1.65 ± 0.68 | 2.10 ± 0.55 |
| Fengshiping | 7.5 | 0.74 ± 0.12 | 0.66 ± 0.074 | 0.83 ± 0.13 | 0.77 ± 0.27 | 1.11 ± 0.45 | 1.34 ± 0.53 | 1.91 ± 0.61 |
| Fengshiping | 15 | 0.80 ± 0.24 | 0.62 ± 0.13 | 0.76 ± 0.18 | 0.49 ± 0.17* | 0.73 ± 0.34* | 1.00 ± 0.48* | 1.38 ± 0.67* |
| Fengshiping | 30 | 0.75 ± 0.19 | 0.67 ± 0.19 | 0.87 ± 0.28 | 0.63 ± 0.22 | 0.73 ± 0.34* | 0.82 ± 0.43** | 1.05 ± 0.53** |
| Tripterygium | 5 | 0.72 ± 0.11 | 0.68 ± 0.16 | 0.91 ± 0.18 | 0.66 ± 0.23 | 0.88 ± 0.29 | 1.03 ± 0.36* | 1.37 ± 0.33* |
| hypoglaucum | | | | | | | | |
| (Levl.) Hutch. | | | | | | | | |
| prednisone | 0.01 | 0.64 ± 0.14 | 0.64 ± 0.16 | 0.50 ± 0.26 | 0.46 ± 0.25 | 0.72 ± 0.46* | 0.87 ± 0.46** | 1.28 ± 0.69* |

| | Dose | Swelling degree(Δcm) | | | | | |
|---|---|---|---|---|---|---|---|
| Group | (g/kg) | 18 d | 20 d | 22 d | 24 d | 26 d | 28 d |
| Control | — | 2.18 ± 0.44 | 2.05 ± 0.46 | 2.00 ± 0.46 | 2.04 ± 0.57 | 1.92 ± 0.65 | 1.83 ± 0.67 |
| Fengshiping | 7.5 | 1.74 ± 0.73 | 1.81 ± 0.55 | 1.81 ± 0.52 | 1.77 ± 0.55 | 1.65 ± 0.55 | 1.55 ± 0.49 |
| Fengshiping | 15 | 1.32 ± 0.59** | 1.28 ± 0.58** | 1.34 ± 0.61* | 1.33 ± 0.67* | 1.20 ± 0.64* | 1.08 ± 0.58** |
| Fengshiping | 30 | 0.95 ± 0.50** | 0.87 ± 0.51** | 0.95 ± 0.54** | 0.89 ± 0.59** | 0.90 ± 0.57** | 0.86 ± 0.51** |
| Tripterygium | 5 | 1.47 ± 0.43** | 1.50 ± 0.43** | 1.49 ± 0.43* | 1.42 ± 0.53* | 1.40 ± 0.56* | 1.32 ± 0.57 |
| hypoglaucum | | | | | | | |
| (Levl.) Hutch. | | | | | | | |
| prednisone | 0.01 | 1.18 ± 0.7**6 | 1.03 ± 0.67** | 1.05 ± 0.69* | 0.90 ± 0.64** | 0.86 ± 0.65** | 0.85 ± 0.59** |

Comparing to the control group *P < 0.05, **P < 0.01 (the signs have the same meaning in the following tables)

TABLE 1.2
The effect of the Fengshiping on the swelling degree of the left anklejoint and footafter the
injection of FCA in the rat AA model (\overline{X} ± S)

| | Dose | Swelling degree (Δcm) | | | | | |
|---|---|---|---|---|---|---|---|
| Group | (g/kg) | 2 d | 9 d | 12 d | 14 d | 16 d | 18 d |
| Control | — | 0.14 ± 0.05 | 0.06 ± 0.10 | 0.34 ± 0.36 | 0.80 ± 0.52 | 1.43 ± 0.67 | 1.36 ± 0.61 |
| Fengshiping | 7.5 | 0.18 ± 0.06 | 0.10 ± 0.14 | 0.26 ± 0.36 | 0.82 ± 0.52 | 1.31 ± 0.64 | 1.28 ± 0.71 |
| Fengshiping | 15 | 0.15 ± 0.08 | 0.02 ± 0.06 | 0.13 ± 0.10* | 0.37 ± 0.31* | 0.90 ± 0.56* | 0.79 ± 0.60* |
| Fengshiping | 30 | 0.18 ± 0.09 | 0.06 ± 0.06 | 0.16 ± 0.08* | 0.29 ± 0.20** | 0.49 ± 0.41** | 0.33 ± 0.29** |
| Tripterygium | 5 | 0.16 ± 0.07 | 0.01 ± 0.07 | 0.11 ± 0.10 | 0.44 ± 0.19** | 0.87 ± 0.56* | 0.84 ± 0.67* |
| hypoglaucum | | | | | | | |
| (Levl.) Hutch. | | | | | | | |
| prednisone | 0.01 | 0.20 ± 0.06 | 0.08 ± 0.08 | 0.21 ± 0.16 | 0.44 ± 0.43 | 0.99 ± 0.63 | 0.84 ± 0.74* |

| | Dose | Swelling degree (Δcm) | | | | |
|---|---|---|---|---|---|---|
| Group | (g/kg) | 20 d | 22 d | 24 d | 26 d | 28 d |
| Control | — | 1.28 ± 0.57 | 1.38 ± 0.64 | 1.35 ± 0.75 | 1.20 ± 0.78 | 1.12 ± 0.63 |
| Fengshiping | 7.5 | 1.33 ± 0.71 | 1.31 ± 0.73 | 1.27 ± 0.73 | 1.16 ± 0.73 | 1.07 ± 0.65 |
| Fengshiping | 15 | 1.74 ± 0.57* | 1.92 ± 0.61* | 0.95 ± 0.64* | 0.88 ± 0.58* | 1.83 ± 0.55 |
| Fengshiping | 30 | 0.27 ± 0.30** | 0.34 ± 0.31** | 0.32 ± 0.33** | 0.31 ± 0.32** | 0.34 ± 0.32** |
| Tripterygium | 5 | 0.82 ± 0.65* | 0.89 ± 0.70* | 0.80 ± 0.67* | 0.83 ± 0.68 | 0.75 ± 0.69 |
| hypoglaucum | | | | | | |
| (Levl.) Hutch. | | | | | | |
| prednisone | 0.01 | 0.82 ± 0.72* | 0.79 ± 0.74* | 0.75 ± 0.67** | 0.68 ± 0.64* | 0.71 ± 0.67 |

1.3 The effect of the Fengshipng on the body weight of the AA
rats (X ± S)

| | | Body weight change(g) | | |
|---|---|---|---|---|
| | Dose | | BW at 1 month | |
| Group | (g/kg) | Initiative BW | later | BW change |
| Control | — | 228 ± 34 | 231 ± 52 | 3 |
| Fengshiping | 7.5 | 229 ± 34 | 220 ± 46 | −9 |
| Fengshiping | 15 | 223 ± 40 | 232 ± 34 | 9 |
| Fengshiping | 30 | 224 ± 37 | 256 ± 60 | 32 |
| Tripterygium | 5 | 226 ± 45 | 230 ± 43 | 4 |
| hypoglaucum | | | | |
| (Levl.) Hutch. | | | | |
| prednisone | 0.01 | 264 ± 55 | 244 ± 31 | −21 |

TABLE 1.4
The effect of the Fengshiping on the organ weight of the immune
system in the AA rats (prevention experiment)(X ± S)

| | Dose | Organ index [(organ weight/body weight)/100] | | | |
|---|---|---|---|---|---|
| Group | (g/kg) | Liver | Spleen | Thymus | adrenal gland |
| Control | — | 3.92 ± 0.65 | 0.34 ± 0.10 | 0.098 ± 0.040 | 0.027 ± 0.01 |
| Fengshiping | 7.5 | 3.73 ± 0.29 | 0.31 ± 0.09 | 0.078 ± 0.038 | 0.027 ± 0.008 |
| Fengshiping | 15 | 3.48 ± 0.32 | 0.38 ± 0.10 | 0.100 ± 0.034 | 0.023 ± 0.005 |
| Fengshiping | 30 | 3.38 ± 0.28* | 0.44 ± 0.12* | 0.100 ± 0.032 | 0.022 ± 0.007 |
| Tripterygium | 5 | 3.21 ± 0.30** | 0.36 ± 0.05 | 0.052 ± 0.011** | 0.026 ± 0.009 |
| hypoglaucum | | | | | |
| (Levl.) Hutch. | | | | | |
| prednisone | 0.01 | 3.04 ± 0.20** | 0.32 ± 0.08 | 0.050 ± 0.060** | 0.020 ± 0.004* |

1.2 The Therapeutic Effect on the AA of the Invented Medicine

50 male SD rats were divided into 5 groups at random. The model building was same to the prevention experiment, but the correspondent medicines were drenched 13 days after the injection of the FCA. The medicines were drenched once a day for 2 weeks. The swelling degree (Δcm) was the difference of the perimeters between the value of first administration day and the other days. (Se the result in table 1.5, 1.6) The major organs' weight is showed in table 1.7.

TABLE 1.5
The therapeutic effect of Fengshiping on the swelling degree of
The left anklejoint and foot in the AA rats (\overline{X} ± S)

| | Dose | Swelling degree (Δcm) | | | |
|---|---|---|---|---|---|
| Group | (g/kg) | 1 d | 2 d | 4 d | 6 d |
| Control | — | 1.81 ± 0.27 | 1.92 ± 0.19 | 2.12 ± 0.22 | 2.16 ± 0.27 |
| Fengshiping | 7.5 | 1.68 ± 0.50 | 1.64 ± 0.54 | 1.70 ± 0.57 | 1.82 ± 0.61 |
| Fengshiping | 15 | 1.44 ± 0.41* | 1.51 ± 0.36** | 1.65 ± 0.34** | 1.74 ± 0.31** |
| Fengshiping | 30 | 1.50 ± 0.56 | 1.48 ± 0.41** | 1.51 ± 0.44** | 1.59 ± 0.51** |
| prednisone | 0.01 | 1.78 ± 0.51 | 1.70 ± 0.51 | 1.63 ± 0.50* | 1.58 ± 0.50** |
| | Dose | Swelling degree (Δcm) | | | |
| Group | (g/kg) | 8 d | 10 d | 12 d | 14 d |
| Control | — | 1.92 ± 0.32 | 1.87 ± 0.34 | 1.92 ± 0.39 | 1.78 ± 0.44 |
| Fengshiping | 7.5 | 1.67 ± 0.68 | 1.60 ± 0.71 | 1.61 ± 0.77 | 1.58 ± 0.71 |
| Fengshiping | 15 | 1.46 ± 0.37** | 1.48 ± 0.30* | 1.28 ± 0.37** | 1.22 ± 0.38** |
| Fengshiping | 30 | 1.29 ± 0.58** | 1.29 ± 0.65** | 1.26 ± 0.67** | 1.20 ± 0.68* |
| prednisone | 0.01 | 1.27 ± 0.46** | 1.09 ± 0.54** | 0.94 ± 0.50** | 0.94 ± 0.42** |

TABLE 1.6
The therapeutic effect of Fengshiping on the swelling degree of
the right anklejoint and foot in the AA rats (X ± S)

| | Dose | Swelling degree (Δcm) | | | | | | |
|---|---|---|---|---|---|---|---|---|
| Group | (g/kg) | 2 d | 4 d | 6 d | 8 d | 10 d | 12 d | 14 d |
| Control | — | 0.36 ± 0.26 | 0.45 ± 0.25 | 0.55 ± 0.34 | 0.47 ± 0.29 | 0.48 ± 0.25 | 0.46 ± 0.31 | 0.40 ± 0.36 |
| Fengshiping | 7.5 | 0.12 ± 0.25 | 0.34 ± 0.32 | 0.48 ± 0.41 | 0.28 ± 0.38 | 0.35 ± 0.30 | 0.30 ± 0.29 | 0.30 ± 0.35 |
| Fengshiping | 15 | 0.21 ± 0.18 | 0.38 ± 0.27 | 0.44 ± 0.33 | 0.21 ± 0.33* | 0.19 ± 0.45* | 0.06 ± 0.31** | −0.06 ± 0.34** |
| Fengshiping | 30 | 0.10 ± 0.48 | 0.06 ± 0.28** | 0.11 ± 0.24** | 0.06 ± 0.27** | 0.02 ± 0.39** | 0.05 ± 0.38* | −0.02 ± 0.41** |
| prednisone | 0.01 | 0.10 ± 0.13* | 0.15 ± 0.28* | 0.11 ± 0.25** | −0.08 ± 0.34** | −0.13 ± 0.28** | −0.26 ± 0.36** | −0.33 ± 0.39** |

n = 10,
comparing with the control group, *P < 0.05, **P < 0.01

TABLE 1.7
The effect of the Fengshiping on the organ weight of the immune
system in the AA rats (X ± S)

| | Dose | Organ index [(organ weight/body weight)/100] | | | |
|---|---|---|---|---|---|
| Group | (g/kg) | Liver | Spleen | Thymus | adrenal gland |
| Control | — | 0.35 ± 0.23 | 0.35 ± 0.061 | 0.073 ± 0.014 | 0.026 ± 0.0071 |
| Fengshiping | 7.5 | 3.21 ± 0.52 | 0.33 ± 0.091 | 0.071 ± 0.026 | 0.024 ± 0.0085 |
| Fengshiping | 15 | 3.40 ± 0.54 | 0.36 ± 0.014 | 0.067 ± 0.022 | 0.023 ± 0.0048 |
| Fengshiping | 30 | 2.79 ± 0.43 | 0.32 ± 0.083 | 0.069 ± 0.029 | 0.023 ± 0.0072 |
| Tripterygium | 5 | 3.92 ± 0.59 | 0.35 ± 0.100 | 0.075 ± 0.034 | 0.027 ± 0.0060 |
| hypoglaucum | | | | | |
| (Levl.) Hutch. | | | | | |
| prednisone | 0.01 | 3.52 ± 0.35 | 0.28 ± 0.047* | 0.05 ± 0.011** | 0.02 ± 0.0043* |

The data showed in the table 1.1, 1.2, 1.3, 1.5 and 1.6 proved that the Fengshiping could strongly inhibit the primary and secondary injury caused by FCA, whenever the medicine was drenched at the beginning of the FCA injection or 2 weeks after the FCA injection. The experiments proved that the Fengshiping had both the preventing and the therapeutic effect. By comparing the effect of Fengshiping on the swelling degree in the anklejoint and foot, we found that the Fengshiping could inhibit the specific immunoswelling in the anklejoint better than the nonspecific immunoswelling in the foot of rats. This result indicated that the main effect of Fengshiping was inhibiting the immunity inflammatory reaction.

The data in the table 1.3, 1.4 and 1.7 showed that the AA rats had no obvious BW increase during the period of the experiment. In the group drenched of the Fengshiping on the effective dosage, the rats still had BW increase. In the groups of prednisone and preventing, the BW of rats had decreased, while the thymus and adrenal gland were atrophied. In the group of tripterygium hypoglaucum (Levl.) Hutch, the thymus had thrinked yet. But in the 3 groups drenched with the different dosage of Fengshiping, on atrophy of the thymus and adrenal gland were observed.

1.3 The Pathologic Change of the AA after the Treatment of the Invented Medicine in Rats

45 SD rats, 180±20 g weight, were divided into 6 groups. After the AA caused by FCA appeared, all the rats were drenched with Fengshiping solution by gastic injection for 5 days once a day. 1 hour after the last administration, the joint index of the rats was measured and calculated. The secondary injuried postpedes' joints on the opposite of the FCA injectiont were taken off and soaked in the formaldehyde. After the tissues were HE tinted, the pathological change of the synovium and cartilage were observed and recorded. The data were showed in table 1.8.

1.8 The effect of Fengshiping on the AA joint index in
the rats (X ± S)

| | Dose | Rat | |
|---|---|---|---|
| Group | (g/kg) | number | Joint index |
| Control | — | 8 | 0** |
| AA model | — | 7 | 6.2 ± 0.49 |
| Fengshiping | 7.5 | 9 | 4.86 ± 0.90** |
| Fengshiping | 15 | 7 | 4.71 ± 0.95** |
| Fengshiping | 30 | 7 | 4.56 ± 1.13** |
| Glucosidorum Tripterygll Totorum | 0.006 | 7 | 4.57 ± 0.79** |

Comparing with the model group **P < 0.01

The joint index was the sum of the inflammatory scores of the four limbs. According to the degree of inflammatory, each limb was evaluated on the criteria as following: normal (0), red without swelling (1), red and swelling (2), seriously swelling (3), deforming and tetanus (4).

Observed from the microscope, the joint synovial membranes of the rat posterior limb were hyperplasia in the model group; and the collagen fiber had increased; there was infiltration of lymphocytes and plasma cells in the tissue. The obvious granuloma had formed. The synovial cells had degenerated and the cytochylema had been tinted red; the caryon had been pycnosis; the epithelium had exfoliated in some part of the synovial membrane. The cartilage turned atrophy; the surface of it was rough and some of the chondrocytes had proliferated lightly.

After the treatment of the Fengshiping, the inflammation of the joint synovial membrane was inhibited, more collogen fiber was produced; less synovial cells exfoliated; the cells on the surface of the cartilage had proliferated and the surface had turned smooth. The cartilage was on the recovering condition.

### EXPERIMENTAL EXAMPLE 2

The effect of Fengshiping on the delayed hypersensitivity reaction (DTH) caused by 2,4-DNFB in the ear of the mouse 50 NIH mice, half male and half female, were divided into 5 groups. Each mouse was led to hypersensitivity reaction by using the 1% DNFB acetone solution on the dosage of 0.025 ml at the right place of the abdomen where the pilus had been cut yet. Using the same solution on the same place was to enhance the hypersensitivity reaction on the third day. On the fifth day, all the mice were smeared with the 1% DNFB edible oil solution at the mice's right ears on the dosage of 0.01 ml each. 24 hours later, all the mice were killed. The mouse's 2 ears were weighted by the torsion balance and the difference of the 2 ears was recorded as the DTH degree caused by the DNFB. The experiment was carried out on the different immune and administration processes.

2.1 The Effect on the DTH by the Full Course Administration

The immune and administration processes is as following:

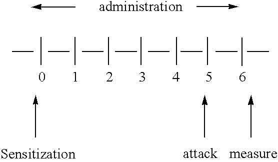

TABLE 2.1
The effect of Fengshiping on the DTH caused by DNFB in the NIH mouse (X ± S)

| | dose | Administration time | | | Percent of inhibition | |
|---|---|---|---|---|---|---|
| group | (g/kg) | (day) | Mice number | Percent of ear swelling | (%) | P value |
| control | | | 10 | 34.20 ± 3.77 | | |
| Fengshiping | 27 | 0˜5 | 10 | 26.24 ± 3.34 | 23.3 | <0.01 |
| Fengshiping | 40 | 0˜5 | 10 | 12.99 ± 4.96 | 62.0 | <0.01 |
| Fengshiping | 60 | 0˜5 | 10 | 10.43 ± 7.53 | 69.5 | <0.01 |
| cortisumman | 0.003 | 0˜5 | 10 | 13.93 ± 4.41 | 59.3 | <0.01 |
| control | | | 10 | 42.43 ± 5.28 | | |
| Fengshiping | 40 | −2˜0 | 10 | 31.50 ± 10.52 | 25.0 | <0.01 |
| Fengshiping | 40 | −2˜2 | 10 | 30.88 ± 7.92 | 27.2 | <0.01 |
| Fengshiping | 40 | −2˜5 | 10 | 21.07 ± 4.62* | 50.3 | <0.01 |
| Fengshiping | 40 | 5˜6 | 10 | 32.00 ± 9.37 | 41.7 | <0.01 |
| cyclophosphane | 0.05 | −2˜2 | 10 | 39.40 ± 10.78 | 8.1 | >0.05 |
| cyclophosphane | 0.05 | −2˜0 | 10 | 37.47 ± 6.71 | 11.7 | >0.05 |
| control | | | 10 | 38.50 ± 4.67 | | |
| cyclophosphane | 0.1 × 3 | 0, 2, 4 day once a day | 10 | 23.00 ± 7.65 | 40.3 | <0.01 |
| cyclophosphane | 0.25 | −3 d | 10 | 41.84 ± 7.75 | −8.7 | |
| Fengshiping | 60 | 0˜4 | 10 | 27.20 ± 10.20 | 29.4 | <0.01 |
| cyclophosphane + Fengshiping | 0.25 + 60 | −3, 0˜4 | 10 | 38.07 ± 6.65 | 1.1 | |

*comparing with the other groups P < 0.05 [symbol] P < 0.01

According to the data showed in table 2.1, it indicated that the Fengshiping had a obvious inhibiting effect on the DTH caused by DNFB. There was a significant relationship between the dosage and the effect. The inhibiting activity increases when the dosage adds. The inhibiting percent could reach 69.5% on the dosage of 60.9 g/kg.

2.2 The Effect on the DTH of the Different Administration Time

The immune and administration processes and the correspondent results had been showed in middle and bottom parts of the table 2.1. According to the results showed in the table 2.1, all the administration ways could significantly inhibit the DTH of the mouse in spite of the administration beginning from the 2 days before the sensitization and ending at the sensitization, or beginning from the 2 days before the sensitization and ending 2 days after the sensitization, or beginning from the 2 days before the sensitization and ending 5 days after the sensitization, or beginning before the attack and ending after the attack. But the administration way that began 2 days before the sensitization and ended 5 days after the sensitization had the most powerful inhibiting activity. It indicated that the Fengshiping could inhibit the DTH by a compound mechanism that it could inhibit the cells participant in the early period of the DTH, the effector cells in the advanced period and the cells related to the DTH in the middle period. This mechanism was different from that of the cyclophosphane. On a small dosage, the cyclophosphane didn't affect the DTH, if its administration way was beginning from the 2 days before the sensitization and ending at the sensitization day or 2 days after the sensitization day.

Based on the bottom part of the table 2.1, if a high dosage of cyclophosphane was drenched to the mouse in one time 3 days before the sensitization, the function of the Th cells would turn sthenic because of the powerful inhibition on the Ts cells. The DTH in the mouse would be enhanced. If the cyclophosphane was used with the Fengshiping on this administration way, it could lower the inhibiting activity of Fengshiping. This result indicated that the Fengshiping have a different machnizm to the cyclophosphane in the control of DTH. The Fengshiping maybe had a higher activity in inhibiting the TH cells.

### EXPERIMENTAL EXAMPLE 3

### The Effect on the Humoral Immunity

3.1 The Effect on the Produce of the Hemolysin Antibody Caused by the Chick RBC

190 mice, 18-22 g weight, half male and half female, were divided into 19 groups. Each mouse was immunized with 5% CRBC solution 0.2 ml. The Fengshiping solutions were drenched at the different times. 7 days after the immunization, all the mice were sampled the blood from the eyes. Then the blood samples were diluted and measured the level of the hemolysin antibody. The results were showed in table 3.1, 3.2 and 3.3.

TABLE 3.1
The effect of Fengshiping on the produce of the hemolysin
antibody in the NIH mouse (X ± S)

| | dose | Administration | Mouse | Hemolysin | Inhibiting | |
|---|---|---|---|---|---|---|
| group | (g/kg) | time | number | value | percent (%) | P value |
| control | | | 10 | 169.0 ± 62.0 | | |
| Fengshiping | 18 | 0˜7 | 10 | 46.0 ± 15.6 | 72.8 | <0.01 |
| Fengshiping | 27 | 0˜7 | 10 | 35.4 ± 12.0 | 79.1 | <0.01 |
| Fengshiping | 40 | 0˜7 | 10 | 28.2 ± 5.9 | 83.3 | <0.01 |
| Fengshiping | 60 | 0˜7 | 10 | 16.7 ± 3.0 | 90.1 | <0.01 |
| Tripterygium | 13.3 | 0˜7 | 10 | 121.0 ± 88.0** | 28.4 | <0.015 |
| hypoglaucum (Levl.) | | | | | | |
| Hutch. | | | | | | |
| cyclophosphane | 0.02 | 0˜7 | 10 | 35.0 ± 12.0 | 79.3 | <0.01 |

**comparing with the Fengshiping (40 g/kg) group P < 0.01

TABLE 3.2
The effect of Fengshiping on the produce of the hemolysin
antibody in the ICR mouse (X ± S)

| | dose | Administration | Mouse | | inhibiting | P |
|---|---|---|---|---|---|---|
| group | (g/kg) | time | number | Hemolysin value | percent (%) | value |
| control | — | — | 10 | 124.70 ± 42.60 | | |
| Fengshiping | 12 | 0˜7 | 10 | 75.00 ± 53.10 | 39.9 | <0.05 |
| Fengshiping | 18 | 0˜7 | 10 | 45.60 ± 22.70 | 63.4 | <0.01 |
| Fengshiping | 27 | 0˜7 | 10 | 29.10 ± 22.10 | 76.8 | <0.01 |
| Fengshiping | 40 | 0˜7 | 10 | 28.20 ± 5.30 | 77.4 | <0.01 |
| Tripterygium | 6.0 | 0˜7 | 10 | 143.50 ± 67.90** | | >0.05 |
| hypoglaucum (Levl.) | | | | | | |
| Hutch. | | | | | | |
| cyclophosphane | 0.02 | 0˜7 | 10 | 27.80 ± 6.60 | 77.9 | <0.01 |

**comparing with the Fengshiping (18 g/kg) group P < 0.01

TABLE 3.3
The effect of Fengshiping on the produce of the hemolysin
antibody in the ICR mouse (X ± S)

| | dose | Administration | Mouse | Hemolysin | Inhibiting percent | P |
|---|---|---|---|---|---|---|
| group | (g/kg) | time | number | value | (%) | value |
| control | — | — | 10 | 256.0 ± 26.0 | | |
| Fengshiping | 18 | −7˜7 | 10 | 198.0 ± 50.0 | 22.7 | <0.01 |
| Fengshiping | 18 | −3˜7 | 10 | 156.0 ± 85.0 | 39.1 | <0.01 |
| Fengshiping | 18 | 0˜7 | 10 | 98.0 ± 35.0 | 61.7 | <0.01 |
| cyclophosphane | 0.02 | 0˜7 | 10 | 25.0 ± 4.0 | 90.2 | <0.01 |

According to the data in table 3, it indicated that the Fengshiping had a remarkable inhibiting effect on the produce of the hemolysin antibody in the different mouse species and this effect would increase along with the increase of the dosage. There was a certain relationship between the dosage and the effect. The lowest effective dosage was 12 g/kg. Comparing with the same quantity of Tripterygium hypoglaucum (Levl.) Hutch, the Fengshiping had a higher inhibiting activity. Based on the data in table 3.1, the inhibiting activity of Fengshiping was 2.25 times higher than the Tripterygium hypoglaucum (Levl.) Hutch. The inhibiting activity of Tripterygium hypoglaucum (Levl.) Hutch. On the dosage of 13.5 g/kg was weaker than that of the Fengshiping which containing 6 g/kg Tripterygium hypoglaucum (Levl.) Hutch).

3.2 The Effect of the Fengshiping on the Humoral Immunity in the AA Mouse

The NIH mice, 20±2 g weight, were injected with 0.05 ml FCA under the vola skin of the right postpedes. 3 weeks late the AA model mouse builded. The model mice were divided into 6 groups randomly and drenched with the correspondent medicines for 5 days. At the beginning of the administration, all the mice were sensitized with 0.5 ml 10% sheep RBC (SRBC). Five days later, all the mice were killed. Their spleens were taken out and washed by the Hank's liquor to prepare the lymphocyte suspended liquor. The concentration of the cells was adjusted to 2×10⁷/ml. 1 ml lymphocyte suspension, ml 0.2% SRBC and 1 ml 1:30 addiment were added to one test tube. The tube was put in the water bath at 37° C. for 1 hour. Then the tube was centrifugated at 2000 rpm for 5 minutes. The supernatant fluid was separated and tested its sptical density at the 415 nm wavelength on the 722 apeotrophotometer. The value was the represent of PFC quantity.

The other share of the blood samples got from the sensitized mice was separated the serum to test the potency of the antibody. The measured data were recorded on the way of Log 2 value. (See the data in table 3.4)

TABLE 3.4
The effect of Fengshiping on the humoral immunity in the
mouse (X ± S)

| | dose | Mouse | PFC | |
|---|---|---|---|---|
| group | (g/kg) | number | (OD) | IgM(Log2) |
| control | — | 8 | 0.819 ± 0.013# | 6.875 ± 0.641 |
| AA model | — | 10 | 0.940 ± 0.019** | 7.700 ± 0.599* |
| group | | | | |
| fengshiping | 5 | 8 | 0.834 ± 0.012**# | 6.875 ± 0.641# |
| fengshiping | 10 | 8 | 0.834 ± 0.012**# | 6.750 ± 0.886# |
| fengshiping | 20 | 8 | 0.830 ± 0.014**# | 6.375 ± 0.518## |
| Glucosidorum | 0.012 | 10 | 0.835 ± 0.015**# | 6.950 ± 0.597# |
| Tripterygll | | | | |
| Totorum | | | | |

Comparing with the control group *P < 0.05, **P < 0.01; comparing with the model group #P < 0.05, ##P < 0.01

According to the table 3.4, the levels of PFC and IgM in the AA mouse were higher than that of the normal mouse. The Fengshiping could lower the produce of the PFC and IgM in the AA mouse significantly.

### EXPERIMENTAL EXAMPLE 4

### The Effect of the FENGSHIPING on the Passive Cutis Anaphylactic Reaction (PCA) in the Rat

The rats were injected with the egg albumn at 10 mg/kg in the muscle. At the same time, all the rats were injected with 2×10¹⁰ (0.2 ml) bordetella pertussis in the abdominal cavity for sensitization. 2 weeks later, all the rats were killed to sample the blood. All the blood samples were separated for preparing the serum.

60 rats, 150˜200 g, half male and half female, were divided into 6 groups at random. In the light narcosis condition induced by aether, each rat was cut the fleece in the back and injected with the 2 concentrations of anti-egg-album serum 0.1 ml under the skin at the fairless place. The serums were diluted on the rates of 1:5(d1) and 1:10(d2) before the experiment. 48 hours later, all the rats were attacked by intravenous injecting the 0.5% evans blue normal saline solution 1 ml which containing 1 mg egg albumin. 20 minutes later, the rats were killed by decapitation. The rats' back skin were dissected and turned over. According to the dark and light and area of the blue stains exudated from the vessels, all the rats were evaluated by several people. The skin stained by the evens blue were scissored and soaked in 5 ml 0.1% sodium sulfate acetone (7:3) solution for 48 hours. Then it was centrifugated to separate the supernatant liquor. The supernates were measured the optical density at the wavelength 590 nm to calculate the degree of the PCA reaction and the inhibiting percent. The results were shown in table 4.

TABLE 4
The effect of Fengshiping on the PCA in rat (X ± S)

| | dose | value | | absorbancy | |
|---|---|---|---|---|---|
| Group | (g/kg) | d₁ | d₂ | d₁ | d₂ |
| Control | — | 5.60 ± 1.78 | 2.40 ± 2.46 | 0.191 ± 0.129 | 0.096 ± 0.106 |
| Fengshiping | 12 | 7.50 ± 2.51 | 4.20 ± 2.49 | 0.402 ± 0.213* | 0.192 ± 0.175 |
| Fengshiping | 24 | 7.10 ± 2.13 | 4.10 ± 1.79 | 0.310 ± 0.177 | 0.137 ± 0.099 |
| Fengshiping | 48 | 6.00 ± 1.83 | 1.70 ± 1.95 | 0.121 ± 0.109 | 0.024 ± 0.026* |
| Tripterygium hypoglaucum | 8 | 6.11 ± 1.27 | 2.56 ± 1.67 | 0.223 ± 0.122 | 0.074 ± 0.045 |
| (Levl.) Hutch. | | | | | |
| Ketotifen | 0.1 | 2.78 ± 1.64** | 0.67 ± 1.41 | 0.033 ± 0.024** | 0.027 ± 0.019* |

Comparing with the control group *P < 0.05, **P < 0.01

According to the table 4, it indicated that the Fengshiping had a weak effect on the PCA in the rat. Only on a high dosage, the inhibiting effect of Fengshiping was obviously different from that of the control group.

### EXPERIMENTAL EXAMPLE 5

### The Effect of Fengshiping on the Cytokines

5.1 The effect of Fengshiping on the levels of TNF α and IL-2 in the mouse.

60 ICR mice, 1822 g, half male and half female, were divided into 6 groups at random. Each group was drenched of the correspondent medicines including the different dosages of Fengshiping and the other medicines. The medicines were administrated once a day for 10 days. 24 hours after the last administration, the mice were sampled the macrophage and spleen cells from the abdominal cavity in the aseptic condition. The samples were washed with Hank's liquor for 2 times and non-serum RPIM 1640 liquor for 1 time. Then the washed samples were diluted to the suspension with the 5% FCS-RPMI 1640 at the concentration of 2×10⁸/ml. Then the suspensions were added with 10 ng/ml LPS or the 10 ng/ml ConA and cultured in the 5% CO2 condition for 48 hours at 37° C. Then the cultured suspension were measured the TNF α and IL-2 levels on the usual methods.

The Measurement of TNF α

The batten was coated by mouse TNF-α monoclonal antibody. The batten was added with the cultured supernate on the dose of 50 μl/hole. Then the batten was put still for 60 minutes at the room temperature. Then the batten was added with biotin antibody mark at 25° C. for 2 hours. Then the enzyme labeled avidin was added into the batten for 30 minutes. After adding the substrate constant for 30 minutes, the batten was added with the stop liquor. The mixed liquor was measured the OD value at the wavelength of the 450 nm. The content of the TNF-α (ng/ml) was calculated on the data of OD value by the method of standard curve.

The Measurement of the IL-2:

The CTLL cells which was on the logarithmic growth phase and whose growth depends on the IL-2, were adjusted to the suspension at the concentration of 1×10⁵/ml with the 5% FCS-RPMI 1640. Then the 96 hole cell culturing batten were added with the CTLL cell suspension on the quantity of 100 μl/hole. The supernates were added on the quantity of 100 μl/hole and each sample was added to 3 holes. The samples cultured were compared with the different dilutions of standard rHIL-2 and the control sample (culture fluid) to measure the IL-2. All the samples were cultured in the 5% CO2 for 24 hours at 37° C. 6 hours before the end of the culture, all the samples were centrifuged and separated the supernate. Each hole were taken out 110 μl supernate and added with 10 μl MTT. The samples were cultured for 3 hours at 37° C., and then measured the OD at the wavelength 570 nm and 630 nm. The final OD value of the sample was the difference of OD (570 nm) and OD (630 nm).

TABLE 5.1
The effect of Fengshiping on the TNF α nd IL-2 (X ± S)
$IL \text{-} 2 activity = \frac{Sample \overline{OD} - Control ( Culture Fluid ) \overline{OD}}{\begin{matrix}Standard Sample \overline{OD} - Control ( Culture Fluid ) \overline{OD} \times \\ activity of the standard sample \text{(IU/ml)}\end{matrix}}$

| | dose | Mouse | TNF | IL-2 |
|---|---|---|---|---|
| group | (g/kg) | number | (pg/ml) | (IU/ml) |
| Control | — | 10 | 87.80 ± 14.63 | 26.30 ± 4.22 |
| | 12 | 10 | 62.14 ± 13.13** | 16.00 ± 2.89** |
| Fengshiping | 24 | 10 | 58.60 ± 9.63** | 18.80 ± 2.86** |
| | 36 | 10 | 54.40 ± 10.88** | 18.20 ± 2.86** |
| Tripterygium | 8 | 10 | 58.25 ± 10.32** | 16.00 ± 2.88** |
| hypoglaucum | | | | |
| (Levl.) Hutch. | | | | |
| cyclophosphane | 0.02 | 10 | 42.20 ± 9.57** | 10.10 ± 3.00** |

*P < 0.05,
**P < 0.01

According to the data in Table 5.1, it suggested that the Fengshiping have a obvious inhibiting effect on the TNF α. On the dosage of 12 g/kg, the medicine had showed a obvious inhibiting effect. Along with the increase of the dosage, the inhibiting effect increased. But the dosage-effect curve went gently. The Fengshiping had an obvious inhibiting effect on the IL-2, ut no dosage-effect relationship was observed.

5.2 The Effect of Fengshiping on the IL-1, IL-6

70 NIH mice, 18-22 g weight, half male and half female, were divided into 7 groups at random. All the groups were drenched with the correspondent medicines (fengshiping and the other medicines). The medicines were drenched once a day for 10 days. 24 hours after the last administration, all the mice were killed and sampled the macrophage and spleen cells from the abdominal carvity. The IL-1 and IL-6 in the samples were measured.

The Measurement of IL-1:

The macrophages in the abdominal carvity were sampled in the asepsis condition. Then the samples were washed by the Hank's liquor for 2 times and nonserum RPMI1640 liquor for 1 time. Then the clear samples were adjusted to the 4×10⁶/ml cell suspension with 5% FCS-RPMI liquor. 1 ml of the suspension was added to the test tube and cultured at 37° C. for 1 hour. The unadherent cells were abandoned. Then the cultured liquor was added with 5% FCS-RPMI 1640 and LPS (10 ng/ml) to culture. The cells cultured in 5% CO₂ at 37° C. for 72 hours. During the course, the cultured cells were freezed and thawed for several times. The final product was saved at 4° C. The C57 mice were sampled the thymus in the asepsis condition. Then the samples were prepared to the 1×10⁶/ml cell suspension with 5% FCS-RPMI1640.

100 μl supernate separated from the frost thawing liquor and 100 μl cell suspension of thymus were added into the 96-hole flat bottom cell-culture batten. Each sample was cultured in 3 holes and compared with the different dilutions standard rHIL-1 and the control sample (culture fluid). Each hole was added with 2 ng ConA and then the batten was cultured in the 5% CO₂ at 37° C. for 72 hours. 14 hours before the end of the culture, each hole was added with 3H-TdR 0.1μ Ci. The cultured cells were collected with multihead cell-harvesting apparatus and measured the cpm value.

$$IL \text{-} 1 activity = \frac{Sample \overline{cpm} - Control ( Culture Fluid ) \overline{cpm}}{Standard Sample \overline{ cpm} - Control ( Culture Fluid ) \overline{cpm}} \times activity of the standard ( ng \text{/} ml )$$

The Measurement of the IL-6:

The spleen cells were sampled in the asepsis condition. Then the samples were washed by the Hank's liquor for 2 times and nonserum RPMI1640 liquor for 1 time. Then the clear samples were adjusted to the 2×10⁶/ml cell suspension with 5% FCS-RPMI liquor. 1 ml of the suspension was added to the round-bottom centrifuge tube. After adding the ConA (10 ng/ml), the samples were cultured in the 5% CO₂ at 37° C. for 72 hour.

The MH60 cells, which grew depending on the IL-6 and were on the logarithmic growth stage, were adjusted to the 1×10⁵/ml cell suspension with the 5% FC-RPMI1640.

The 96-hole flat bottom cell culturing batten was added with the MH60 cell suspension on the quantity of 100 μl/hole and the culturing supernate 25 μl/hole. Then the fluid in each hole was adjusted to the 200 μl with the 5% FCS-RPMI 1640. Each sample was cultured with 3 copies and compared with the different solutions standard rHIL-6 and the pure culturing fluid. The batten was cultured in 5% CO₂ at 37° C. for 72 hours. 6 hours before the end of the culture, the samples were centrifuged. Each hole was sucked out the supernate 110 μl and added the MTT 10 μl. The samples were kept at 37° C. for 3 hours. And then they were measured the OD at the wavelength 570 nm and 630 nm. The final OD value=OD 570 nm-OD 630 nm.

TABLE 5.2
The effect of Fengshiping on the IL-1, IL-6 (X ± S)
$IL \text{-} 6 activity = \frac{SampleOD - Culturing Fluid ControlOD}{\begin{matrix}Standard SampleOD - Culturing Fluid ControlOD \times \\ Sample Dilution \times Activity Of The Standard \text{(IU/ml)}\end{matrix}}$

| | Dose | Mouse | IL-1 | IL-6 |
|---|---|---|---|---|
| Group | (g/kg) | number | (ng/ml) | (IU/ml) |
| Control | — | 10 | 78.7 ± 7.1 | 94.6 ± 6.8 |
| | 7.5 | 10 | 59.3 ± 4.9** | 64.9 ± 4.8** |
| | 15 | 10 | 53.3 ± 5.7** | 60.5 ± 4.3** |
| Fengshiping | 30 | 10 | 54.4 ± 4.8** | 56.0 ± 4.6** |
| | 60 | 10 | 47.0 ± 16.6** | 56.6 ± 6.1** |
| Tripterygium | 5 | 10 | 57.6 ± 4.7** | 65.7 ± 4.9** |
| hypoglaucum | | | | |
| (Levl.) Hutch. | | | | |
| cyclophosphane | 0.02 | 9 | 44.5 ± 7.7 | 49.6 ± 6.7** |

Based on the data in the table 5.2, the Fengshiping had an abvious inhibiting effect on the macrophage in producing of IL-1 and spleen cell in producing IL-6. Along with the increase of the dosage, the effect enhanced too.

5.3 The Effect of Fenghsiping on the Plasma NO in the AA Rat

60 SD rats, 160˜220 g weight, half male and half female, were divided into 6 groups. The rats in the blank control group were injected the NS 0.5 ml under the skin of the right postpede vola. Other rats were injected with the FCA 0.5 ml at the same place of the control group. 18 days later, the AA model was built. Then the rats were drenched the correspondent medicines or the distilled water once a day for 5 days. 3 groups were drenched the solution of Fengshiping on the high, middle and low dilution. The positive group was drenched with Glucosidorum Tripterygll Totorum. The blank control group and the model group were drenched with the distilled water of the same volumn. 1 hour after the last administration, each rat was sampled the blood from the abdominal aorta for 2 ml. The plasma of the blood samples were separated and saved at −70° C. for the measurement. The measurement of NO was done on the direction of the NO reagent. 0.1 ml plasma was added in 0.6 ml reagent C and 0.4 ml double distilled water. After the mixture shaken up, it was added in 0.1 ml reagent D and cultured on the ice for 60 min. Then it was centrifuged at 12000 rpm for 2 min. The supernate was separated. 0.6 ml supernate was mixed with 0.4 ml double distilled water and 0.1 ml reagent A, and then it was cultured in the ice-water for 15 min. Then the mixture was added in reagent B 0.1 ml and put at the room termperature for 1 hour. Then the new mixture was measured the OD at the wavelength 545 nm. Based on the OD value of the sample, the content of NO was calculated on the standard curve. (See the result in table 5.3)

TABLE 5.3
The effect of Fengshiping on the plasma NO level in the AA
rat (X ± S)

| | Dose | Rat | Content of NO | y |
|---|---|---|---|---|
| Group | (g/kg) | number | (μmol/L) | (y = Lgx) |
| Control | — | 8 | 13.55 ± 1.11* | 1.131 ± 0.032 |
| AA model | — | 9 | 17.56 ± 4.15 | 1.235 ± 0.097 |
| Fengshiping | 12 | 7 | 9.83 ± 2.58**^{ΔΔ} | 0.985 ± 0.087 |
| Fengshiping | 24 | 7 | 10.12 ± 1.56**^{ΔΔ} | 1.001 ± 0.067 |
| Fengshiping | 48 | 7 | 10.70 ± 1.51**^{ΔΔ} | 1.026 ± 0.062 |
| Glucosidorum | 0.006 | 7 | 15.25 ± 3.48 | 1.173 ± 0.099 |
| Tripterygll | | | | |
| Totorum | | | | |

Comparing to the model group *P < 0.05, **P < 0.01; comparing to the Glucosidorum Tripterygll Totorum ^{ΔΔ}P < 0.01

Based on the data in table 5.3, the NO level was higher in the model group than in the blank control group. The Fengshiping had an obvious effect on lowering the NO level in the AA rat. The Glucosidorum Tripterygll Totorum had the similar effect but its effect was weaker than that of the Fengshiping.

### EXPERIMENTAL EXAMPLE 6

### The Effect of Fengshiping on the T Lymphocyte, CD₄, CD₈ and NK Cells in the Mouse

6.1 The Effect of Fengshiping on the Transform of Lymphocyte in the Normal Mouse

80 NIH mice, half male and half female, were divided into 8 groups randomly and drenched with the correspondent medicines once a day for 10 days. 24 hours after the last administration, all the mice were killed to sample the spleen cells aseptically. Then the samples were washed by the Hank's liquor for 2 times and nonserum RPMI1640 liquor for 1 time. Then the clear samples were adjusted to the 2×10⁶/ml cell suspension with 5% FCS-RPMI liquor. The 96-hole flat bottom cell culturing batten was added with the cell suspension on the quantity of 100 μl/hole. Each sample was cultured with 3 copies. 2 holes were added in 2 ng ConA each as the stimulating reagent. The other hole was not added in the ConA and kept as the control hole. The batten was cultured in 5% CO₂ at 37° C. for 72 hours. 14 hours before the end of the culture, each hole was added in 3H-TdR 0.1μ Ci. The cells were harvested by the multihead cell harvesting instrument and measured the cpm value. The average value was adopted as the sample's cpm value. The average value and the stimulating index of the different groups were compared directly. The stimulating index was calculated as following:

See the result in table 6.1

TABLE 6.1
The effect of Fengshiping on the lymphacyto transformation
induced by ConA in the mouse (X ± S)
$Stimulating Index = \frac{Stimulated \overline{cpm}}{Control \overline{cpm}}$

| | Dose | Mouse | | Stimulating |
|---|---|---|---|---|
| Group | (g/kg) | number | cpm | index |
| Control | — | 10 | 20433 ± 3579 | 25.87 ± 3.06 |
| | 7.5 | 10 | 13566 ± 1779** | 27.29 ± 7.67 |
| | 15 | 10 | 12708 ± 1692** | 18.04 ± 3.76 |
| Fengshiping | 30 | 10 | 12809 ± 2575** | 16.17 ± 4.37 |
| | 60 | 10 | 12090 ± 1706** | 19.05 ± 3.80 |
| | 2.5 | 10 | 18038 ± 3359 | 17.11 ± 2.60 |
| Tripterygium | 5 | 10 | 12081 ± 1039** | 17.58 ± 4.37 |
| hypoglaucum | | | | |
| (Levl.) Hutch. | | | | |
| Cyclophosphane | 0.02 | 9 | 9922 ± 1145** | 13.66 ± 2.28 |

Comparing to the control group
*P < 0.05,
**P < 0.01

According to the data in table 6.1, it indicated that the Fengshiping had an obvious inhibiting effect on the lymphocyte transformation and there was a dosage-effect relationship.

6.2 The Effect of Fengshiping on the CD₄, CD₈ and NK Cells

The experiment was same to 5.1. 24 hours after the last administration, the spleen cell samples were made into the 2×10⁸/ml cell suspension with 5% FCS-RPIM1640. The quantity of CD₄, CD₈, NK cells and the rate CD₄/CD₈ were measured on the usural method.

The Measurement of CD₄ and CD₈

The spleen cell suspension 50 μl was added on the glass to made the cell smear. The glass had been coated by the polylysine. The T cell of the mouse was set as the positive control sample. The cell smear was enveloped by the serum of the normal mouse after it was fixed by the acetone. Then the enveloped sample was added with the antibody of CD₄ and CD₈ which were marked by the hominine biotin. It was incubated at 37° C. for 2 hours. Then the sample was added with the avidin labeled by enzyme and put still for 10 min. After added with the substrate for 10 min, the mixed sample was washed and dyed with the hematoxylin for 2 min. Then the sample was dyhidrated with the grade-alcohol and enveloped with gelatin-glycetrol. 200 cells in the smear were chosen as the research target under the high power microscope.

$$Content Of Cell = \frac{Dyed cell number}{200} \times 100 \%$$

The Measurement of the NK Cell:

The preparation of the EC cell: The spleen cells were sampled in the asepsis condition. Then the samples were washed by the Hank's liquor for 2 times and nonserum RPMI1640 liquor for 1 time. Then the clear samples were adjusted to the 2×10⁸/ml cell suspension with 5% FCS-RPMI liquor. This cell suspension was used as the EC.

The preparation of the TC cell: The Yack-1 cells, which were sensitive to the mouse NK cell and on the logarithmic growth phase, were adjusted to the 4×10⁴/ml cell suspension. It was the TC.

Measurement: EC and TC, 100 μl each were added in the 96-hole flat bottom cell culturing batten. Each sample was cultured with 3 copies and set 2 control samples: EC and TC. (EC control: EC100 μl+5% FCS RPMI 1640 100 μl; TC control: TC100 μl+5% FCS RPMI 1640 100 μl). The samples were cultured in 5% CO₂ at 37° C. for 24 hours. 6 hours before the end of the culturing, the samples were centrifuged and sucked out 110 μl supernate each hole. And then the holes were added in the MTT 10 μl. After put at 37° C. for 3 hours, the mixed samples were measured the OD value at the wavelength of 570 nm and 630 nm. The OD of each hole=OD570 nm-OD630 nm.

TABLE 6.2
The effect of Fengshiping on the CD4, CD8, NK cell (X ± S)
$Activity Of NK = ( 1 - \frac{Sample \overline{OD} - EC Control \overline{OD}}{TC Control \overline{OD}} ) \times 100 \%$

| Group | Dose (g/kg) | Mouse number | CD4 (%) | CD8 (%) | CD4/CD8 | NK |
|---|---|---|---|---|---|---|
| Control | — | 10 | 20.80 ± 2.94 | 14.80 ± 2.49 | 1.42 ± 0.18 | 40.13 ± 4.89 |
| | 12 | 10 | 19.14 ± 2.91 | 13.43 ± 2.51 | 1.43 ± 0.08 | 31.94 ± 4.52**^{ΔΔ} |
| Fengshiping | 24 | 10 | 17.30 ± 2.51** | 12.00 ± 2.40 | 1.46 ± 0.16 | 35.36 ± 3.40*^{ΔΔ} |
| | 36 | 10 | 16.30 ± 2.50** | 11.23 ± 2.94** | 1.49 ± 0.20 | 31.06 ± 3.53**^{ΔΔ} |
| Tripterygium | 8 | 10 | 16.25 ± 2.25** | 11.50 ± 2.45 | 1.44 ± 0.18 | 32.20 ± 2.00** |
| hypoglaucum | | | | | | |
| (Levl.) Hutch. | | | | | | |
| Cyclophosphane | 0.02 | 10 | 11.50 ± 2.50** | 4.10 ± 1.20** | 2.91 ± 0.53** | 23.10 ± 3.66** |

Comparing to the control group
*P < 0.05,
**P < 0.01;
comparing to the cyclophosphane
^{ΔΔ}P < 0.01

According to the table 6.2, it ie was a relation between the dzosage and the effect, but the dosage-effect curve was smooth. The effective dosage of Fengshiping on the inhibiting of CD₄ was 24 g/kg. The minimum effective dosage on inhibiting the CD₈ was 36 g/kg. As the rate of CD₄/CD₈, the Fengshiping had no obvious effect. Cyclophosphane had an obvious effect on the inhibiting of the both kind of cells, and the inhibiting effect on the CD₈ was very powerful, which could increase the rate of CD₄/CD₈ magnificently.

As for NK cell, the Fengshiping had a remarkable inhibiting effect, but the dosage-effect relationship was not certain. As the same while, the cyclophosphane had shown an obvious inhibiting effect on the NK cell. On the dosage of 20 mg/kg, the inhibitiong effect of cyclophosphane was significantly different from that of the Fengshiping on the 3 dosages: 12, 24 and 36 g/kg.

6.3 The Effect on the Transformation and Function of the T Lymphacyto in the AA Mouse

NIH mice, 20±2 g weight, were injected with 0.05 ml FCA under the skin of the right postpede vola to build the AA model. The mice in the control group were injected 0.05 ml NS at the same place. 3 weeks later, after the AA model built, all the mice were drenched the correspondent medicines once a day for 5 days. 5 days later, all the mice were sampled the blood to make the blood smear. The smears were dyed by the esterase. Then the smears were observed under the oil immersion lens to calculate the percent of the positive-dyed cells (it represented the content of the T cells in the blood). The mice were sampled the spleen cells in the condition of anaesthesia and then the cell samples were prepared to the single cell suspension. The cell suspension was washed by PBS and then its supernate were abandoned. The rest part was added with blood cytolysate 4 ml. The mixed sample was shaked for 2˜3 min to solute the RBC. After the RBCs were destroyed, the sample was centrifuged to separate and abandon the supernate. The sample without supernate was washed by the luminescence lotion for 2 times. Then it was centrifuged to separate and abandon the supernate. In the next step, the sample was adjusted to the 1×10⁶/ml cell suspension. Each tube was added with 50 μl diluted antibody of CD₄ and CD₈. Then the tubs were cultured at 4° C. for 1 hour. After the culture, the samples were washed with the luminescence lotion for 2 times and added in the fixing fluid 2 ml. After fixing, the samples were filtrated through the 400-mesh screen to the FCA tube. The filtrated samples were analyzed by the flow cytometer (FCM). The result was shown in the table 6.3.

TABLE 6.3
The effect of Fengshiping on the T cell in the AA mouse
(X ± S)

| | Dose | ANAE+ | CD4+ | CD8 | |
|---|---|---|---|---|---|
| Group | (g/kg) | (%) | (%) | (%) | CD4+/CD8+ |
| Control | — | 50.60 ± 4.25 | 26.13 ± 1.16 | 15.56 ± 0.68 | 1.68 ± 0.03 |
| AAmodel | — | 49.00 ± 4.22^{▴} | 32.56 ± 2.87** | 13.59 ± 1.03** | 2.49 ± 0.16** |
| Fengshiping | 7.5 | 49.13 ± 4.03^{▴} | 27.30 ± 1.76##^{▴} | 15.98 ± 1.11##^{▴} | 1.71 ± 0.04##^{▴} |
| | 15 | 49.31 ± 3.29^{▴} | 27.96 ± 1.67##^{▴} | 16.23 ± 1.27##^{▴} | 1.73 ± 0.05##^{▴} |
| | 30 | 48.56 ± 3.23^{▴} | 26.75 ± 1.94##^{▴} | 15.58 ± 1.29##^{▴} | 1.72 ± 0.04##^{▴} |
| Glucosidorum | 0.012 | 48.88 ± 2.89^{▴} | 27.88 ± 1.99##^{▴} | 16.33 ± 1.31##^{▴} | 1.70 ± 0.03##^{▴} |
| Tripterygll | | | | | |
| Totorum | | | | | |

n = 8,
comparing with the control group *P < 0.05, **P < 0.01;
comparing with the model group #P < 0.05, ##P < 0.01;
comparing with the control group ^{▴}P > 0.05

According to the data in table 6.3, there was no significant difference in the different groups on the ANAE positive cell. But in AA mouse, the increase of the CD₄ was significant, while the decrease of CD₈ was significant too. So the rate of CD₄/CD₈ had a remarkable increase. The result indicated that the Fengshiping could adjust the CD₄, CD₈ and CD₄/CD₈ to the normal range.

### EXPERIMENTAL EXAMPLE 7

### The Effect of Fengshiping on the Phagocytic Function of the Macrophage in the Mouse Abdominal Carvity

50 NIH mice, 18˜22 g weight, half male and half female, were divided into 5 groups and drenched with the correspondent medicine solutions on the same volumn. The administration was once a day for 7 days. 1 hour after the last administration, all the mice were injected with 0.2 ml 10% chick RBC into the abdominal carvity. 4 hours later, all the mice were killed and sampled the fluid in the abdomincal carvity. The liquor samples were dropped on the glass and counted the number of the macrophage which had phagocytized the CRBC and the number of the CRBC in one macrophage. (See the result in table 7)

TABLE 7
The effect of Fengshiping on the CRBC phagocytosis function
of the macrophage in ICR mouse abdominal cavity (X ± S)

| | dose | Mouse | Percent | phagocytosis |
|---|---|---|---|---|
| group | (g/kg) | number | of phagocytosis (%) | index |
| Control | — | 10 | 25.75 ± 9.40 | 1.28 ± 0.20 |
| Fengshiping | 27 | 10 | 33.20 ± 12.77 | 1.46 ± 0.36 |
| Fengshiping | 40.5 | 10 | 35.20 ± 10.16 | 1.21 ± 0.20 |
| Fengshiping | 60.9 | 10 | 37.78 ± 20.14 | 1.53 ± 0.32 |
| dexamethasone | 0.005 | 10 | 8.33 ± 10.13* | 1.10 ± 0.18 |

*P < 0.05

According to the table 7, the Fengshiping had no obvious effect on the phagocytosis function of the macrophage in the mouse abdominal cavity.

### EXPERIMENTAL EXAMPLE 8

### The Effect of Fengshiping on the Hyperfunction of the Capillary Permeability in the Mouse Abdominal Cavity

90 NIH mice, 18˜22 g weight, half male and half female, were divided into 9 groups and drenched with the correspondent medicine solutions of the same volumn. The medicines were drenched once a day for 3 days or just 1 time. 1 hour after the last administration, each mouse were injected with 0.7% HAC-NS solution into the abdominal cavity. At the same time, each mouse was injected with the 0.5% Evans blue-NS solution into the vessel on the dose of 0.1 ml/10 g. 30 min later; all the mice were killed by cervical disjoint. The abdominal cavity was opened and washed by the 5 ml NS. The NS used was collected and adjusted to 8 ml by the pure NS as the sample. The samples were centrifuged at 3000 rpm to get the supernate. The supernate was measured the OD at the wavelength at 590 nm. (See the result in table 8)

TABLE 8
The effect of Fengshiping on the hyperfunction of the
capillary permeability induced by the acetic acid in the mouse
abdominal cavity (X ± S)

| | Dose | | | Leakage of the tincture | |
|---|---|---|---|---|---|
| Group | (g/kg) | Administration | Mouse number | (OD) | P value |
| Control | — | — | 10 | 0.29 ± 0.13 | |
| Fengshiping | 27 | qd × 1 | 10 | 0.26 ± 0.14 | >0.05 |
| Fengshiping | 40 | qd × 1 | 10 | 0.25 ± 0.10 | >0.05 |
| Fengshiping | 60 | qd × 1 | 10 | 0.25 ± 0.09 | >0.05 |
| Control | — | — | 10 | 0.28 ± 0.15 | |
| Fengshiping | 27 | qd × 3 | 10 | 0.25 ± 0.12 | >0.05 |
| Fengshiping | 40 | qd × 3 | 10 | 0.18 ± 0.10 | <0.05 |
| Fengshiping | 60 | qd × 3 | 10 | 0.15 ± 0.13 | <0.05 |
| dexamethasone | 0.15 | qd × 3 | 10 | 0.11 ± 0.07 | <0.01 |

According to the data in table 8, it indicated that Fengshiping could obviously inhibit the hyperfunction of the capillary permeability induced by the acetic acid in the mouse abdominal cavity if it was drenched for 3 days continuously. If the medicine was drenched for just 1 time, the inhibiting effect was not obvious.

### EXPERIMENTAL EXAMPLE 9

### The Effect of Fengshiping on the Pleuritis Exudation and the Inflammatory Cell Aggregation Induced by the Carrageenan

The mice were divided into 5 groups at random and injected with 0.5% Evans blue NS solution into the caudal vein on the dosage of 0.1 ml/10 g. Then the mice were injected with the 0.03 ml 1% carrageenan in the right chest cavity with the special syring niddle. 4 hours and 32 hours after the injection, the correspondent mice were killed and opend the obdominal cavity to expose the diaphragm. 2 ml of the lotion were injected to the chest cavity by 2 times with a 1 ml injector. The lotion was collected and saved in a test tube. 20 μl of the lotion collected was added into the 400 μl WBC dilution. The WBC in the mixed dilution was counted under the microscope. The rest of the lotion was centrifuged at 3000 rpm for 10 min. The supernate of the lotion was measured the OD at the wavelength of 600 nm. The OD value of the sample should be corrected with the correspondent OD value of the pure lotion. (See the result in table 9)

TABLE 9
The effect of Fengshiping on the inflammatory cell aggregation
induced by the carrageenan (X ± S)

| | Dose | WBC number(2 × 105) | | Tincture exudation (OD) | |
|---|---|---|---|---|---|
| Group | (g/kg) | 4 h | 32 h | 4 h | 32 h |
| Control | — | 46.0 ± 6.9 | 16.0 ± 9.6 | 0.156 ± 0.066 | 0.109 ± 0.019 |
| Fengshiping | 27 | 26.8 ± 4.5* | 14.2 ± 8.0 | 0.121 ± 0.062 | 0.116 ± 0.031 |
| Fengshiping | 40.5 | 10.9 ± 4.0** | 17.3 ± 4.6 | 0.100 ± 0.048 | 0.153 ± 0.032 |
| Fengshiping | 60 | 8.0 ± 5.5** | 6.6 ± 4.7* | 0.129 ± 0.066 | 0.092 ± 0.051 |
| dexamethasone | 0.05 | 12.7 ± 10.2** | 4.4 ± 4.0* | 0.085 ± 0.045 | 0.063 ± 0.017 |

*P < 0.05,
**P < 0.01

According to the table 9, it indicated that the Fengshiping had an obvious inhibiting effect on the inflammatory cell aggregation. The effect was powerful at the early stage. The regression equation on the data of the fourth hour was as following: y=44.13−2.01x, r=−0.9625. The effect on the late stage was weak. At the high dosage of 20 g/kg, the medicine could affect the aggregation of the WBC. But it had no obvious effect on the pleuritis exudation.

### EXPERIMENTAL EXAMPLE 10

### Effect on Aggregation of Leucocyte in Rats' CMC Sac

Sixty four SD rats, 150-180 g weight, half male and half female, were randomly divided into 8 groups, which were drenched with the same volume and different dosage of drug liquid once a day, lasting 3 days. A day before experiment, rats were injected with 20 ml 1% CMC solution into the sac at the rat's back caused by 20 ml air injection before the experiment. 3.5 hour and 7.5 hour later, 0.1 ml liquid in the sac was extracted each time, and was colored in 0.01% brilliant cresyl blue solution. leucocyte was counted in the sac liquor under microscope. The results showed in the table 10.

TABLE 10
effect on leucocyte counts of carboxymethyl cellulose sac of
rats with Fengshiping (X ± S)

| | dosage | rats | WBC count(×107/L) | |
|---|---|---|---|---|
| groups | (g/kg) | number | 3.5 hrs | 7.5 hrs |
| control | — | 8 | 9.7 ± 4.2 | 57.7 ± 17.3 |
| Fengshiping | 27 × 1 | 8 | 8.5 ± 3.5 | 39.4 ± 16.5 |
| Fengshiping | 40 × 1 | 8 | 8.7 ± 7.3 | 35.3 ± 23.2 |
| Fengshiping | 60 × 1 | 8 | 6.6 ± 3.3 | 18.1 ± 8.6** |
| Control | — | 8 | 10.97 ± 6.7 | 35.6 ± 11.2 |
| Fengshiping | 27 × 3 | 8 | 15.4 ± 9.7 | 38.6 ± 15.5 |
| Fengshiping | 40 × 3 | 8 | 4.8 ± 3.4** | 18.4 ± 12.2** |
| Fengshiping | 60 × 3 | 8 | 3.0 ± 2.8** | 11.0 ± 9.2* |
| cortisone | 0.1 × 3 | 8 | 14.2 ± 8.0 | 41.7 ± 16.0 |
| Control | — | 8 | 10.9 ± 3.0 | 41.3 ± 6.9 |
| Fengshiping | 18 × 7 | 8 | 6.2 ± 3.0* | 11.4 ± 6.4* |
| Fengshiping | 27 × 7 | 8 | 3.7 ± 1.7** | 6.4 ± 3.1** |
| Fengshiping | 40 × 7 | 8 | 2.5 ± 1.9** | 5.9 ± 3.9** |
| cortisone | 2 mg × 1 | 8 | 1.5 ± 0.7** | 3.0 ± 1.0** |

Compared with control group **P < 0.01

According to the table 10, the Fengshiping could inhibit significantly aggregation of leucocyte in the rats' CMC sac, and the inhibition showed apparent dosage-effect relation, which was stronger as administration time lasted. With administration of continuing seven days, wandering of leucocyte could be inhibited significantly at dosage of 118 g/kg, at the same time, there was also very strong inhibition with cortisone injection into the sac.

### EXPERIMENTAL EXAMPLE 11

### The Effect on Croton Oil-Induced Swelling in the Ears of Mice

60 NIH mice with weight of 18˜22 g, male and female accounting for half and half, were divided into 6 groups, which were drenched with the same volume and different dosage of drug liquid or tragacanth liquid, once a day, lasting 3 days. 1 hour after the final administration, 2% croton oil mixure of 0.02 ml was embrocated uniformly on the both sides of left ears of mice, and after 4 hours, the mice were snapped off its cervical vertebra and put to death. The left and right ears were cut down, then inflammatory and control ears were weighted by certain means. Difference of weight between left and right ears was the swelling extent of ears, results showing in table 11.

TABLE 11
effect on croton oil-induced swelling of the ears of mice with
Fengshiping (X ± S)

| | dosage | rats | Degree of ears' | inhibition | |
|---|---|---|---|---|---|
| Groups | (g/kg) | number | swelling (mg) | rate (%) | P value |
| Control group | — | 10 | 44.38 ± 9.40 | | |
| Fengshiping | 27 | 10 | 39.05 ± 12.33 | 12.00 | >0.05 |
| Fengshiping | 40 | 10 | 36.65 ± 5.83 | 17.64 | <0.05 |
| Fengshiping | 60 | 10 | 34.91 ± 9.71 | 21.34 | <0.05 |
| dexamethasone | 0.003 | 10 | 14.13 ± 5.75 | 68.16 | <0.01 |

It was seen from table 11, that Fengshiping had remarkable inhibition to croton oil-induced swelling of the ears of mice, and had quantity-effect relation, but which curve was gentle and smooth. There was significant inhibition effect at 13.5 g/kg of dosage.

### EXPERIMENTAL EXAMPLE 12

### Effect on Acetic Acid-Induced Twisting Reaction of Mice

60 Kuming mice with weight of 18˜22 g, male and female accounting for half and half, were randomly divided into 6 groups, which were drenched different dosages of drug liquid or Xihuangqi solution. 1 hours after administration, 0.7% HAC saline of 0.2 ml was injected, sc, and the mice were placed in aquarium and oberved the latent period before the twisting reaction of each mouse and the twisting times in 20 minutes, results showing in table 12:

TABLE 12
The effect of Fengshiping on acetic acid-induced twisting
reaction of mice (X ± S)

| | dosage | Rats | | Latent time |
|---|---|---|---|---|
| groups | (g/kg) | numbers | Twisting times | (minute) |
| Control | — | 10 | 34.6 ± 14.1 | 3.13 ± 0.80 |
| Fengshiping | 27 | 10 | 28.2 ± 5.76 | 3.82 ± 0.85 |
| Fengshiping | 40 | 10 | 31.0 ± 18.4 | 3.86 ± 2.00 |
| Fengshiping | 60 | 10 | 20.7 ± 12.3* | 3.95 ± 1.42 |
| Tripterygium | 20 | 10 | 25.1 ± 11.9 | 3.60 ± 0.93 |
| hypoglaucum | | | | |
| (Levl.) Hutch. | | | | |
| morphine | 10 mg/kg | 10 | 0.0 ± 0.0 | 0.00 ± 0.00 |
| hydrochloride | | | | |

It was seen from table 12 that large dose of Fengshiping could delay the latent time before the RAC-induced twisting reaction and significantly reduce the twisting times in 20 minutes, which indicated Fengshiping had the effect of abirritation in some degree.

### EXPERIMENTAL EXAMPLE 13

### Effect on Hemorheology of AA Rats

Each of SD rats, 180±20 g weight, were injected intracutaneously with 0.05 ml Freund's complete adjuvant on the right back foot metatarsal, and developed into adjuvant arthritis models. Each rats of negtive control group were injected intracutaneously with 0.05 ml salin on the right back foot metatarsal. Three weeks after models built, the rats were divided into model group, large, middle, small dosage group, negtive control group and positive control group which was administered with Glucosidorum Tripterygll Totorum. The rats were drenched once a day, lasting 5 days, 1 hours after administration for the last time, and 3 ml blood was taken from abdominal aorta of rats and placed into test tube with 1% heparin as decoagulant, in which the whole blood viscosity was measured at shear rate of 230, 115, 46, 23, 11.5, 5.75 S⁻¹ with NXE-1 cone and plate viscometer. The plasma viscosity was measured with WTP-BII adjustable constant pressure capillary viscosimeter. The haematocrit, erythrocyte aggregation index was measured with centrifugation method of packed cell volume. The rigidity index was calculated from the above-mentioned data. All the results showed in table 13.

TABLE 13
Effect on hemorheology of adjuvant arthritis model rats (X ± S)

| | | | Fengshiping | Fengshiping | Fengshiping | Glucosidorum Tripterygll |
|---|---|---|---|---|---|---|
| Groups | Control group | Model group | (30 g/kg) | (15 g/kg) | (7.5 g/kg) | Totorum (6 mg/kg) |
| whole blood | | | | | | |
| viscosity | | | | | | |
| (mPa · s) | | | | | | |
| 230S-1 | 4.43 ± 0.09 | 4.92 ± 0.15** | 4.56 ± 0.09## | 4.49 ± 0.11## | 4.54 ± 0.16## | 4.66 ± 0.28# |
| 115S-1 | 5.17 ± 0.25 | 5.81 ± 0.19** | 5.33 ± 0.09## | 5.32 ± 0.10## | 5.16 ± 0.14## | 5.60 ± 0.48# |
| 46S-1 | 6.84 ± 0.11 | 7.20 ± 0.18** | 6.56 ± 0.13## | 6.59 ± 0.09## | 6.67 ± 0.14## | 6.70 ± 0.48# |
| 23S-1 | 8.10 ± 0.15 | 8.23 ± 0.38 | 7.95 ± 0.22 | 7.93 ± 0.12 | 7.97 ± 0.14 | 8.02 ± 0.14 |
| 11.5S-1 | 9.35 ± 0.08 | 9.78 ± 0.10** | 9.40 ± 0.08## | 9.45 ± 0.10## | 9.30 ± 0.133 | 9.31 ± 0.12## |
| 6.5S-1 | 11.03 ± 0.14 | 12.66 ± 0.31** | 11.21 ± 0.21## | 11.29 ± 0.19## | 11.60 ± 0.40## | 11.42 ± 0.52## |
| Plasma viscosity | 1.158 ± 0.032 | 1.248 ± 0.040** | 1.161 ± 0.011## | 1.154 ± 0.023## | 1.156 ± 0.018## | 1.158 ± 0.029## |
| (mPa · s) | | | | | | |
| corpuscular | 46.13 ± 2.31 | 41.33 ± 1.12** | 45.10 ± 2.39## | 44.33 ± 1.52## | 45.71 ± 1.04## | 46.03 ± 3.59## |
| volume (%) | | | | | | |
| erythrocyte | 2.49 ± 0.032 | 2.58 ± 0.083* | 2.46 ± 0.066# | 2.49 ± 0.094# | 2.44 ± 0.048## | 2.45 ± 0.091# |
| aggregation index | | | | | | |
| rigidity index | 6.155 ± 0.536 | 7.127 ± 0.557** | 6.506 ± 0.558 | 6.525 ± 0.146 | 6.394 ± 0.200# | 6.621 ± 0.883 |

Compared with negative control group *P < 0.05, **P < 0.01;
compared with model control group #P < 0.05, ##P < 0.01

According to the table 13, hemorheology of AA rats were changed significantly. The whole blood and plasma viscosity increased, haematocrit decreased, aggregation index and rigity index of erythrocyte increased. The Fengshiping could make the above-mentioned indexes of hemorheology improved significantly.

Pharmacological effects of Fengshiping have been proved by the above-mentioned experiments. Many important pharmacological effects of Fengshiping had favorable dosage-effect relation, which implied the best therapeutic effectiveness might be obtained by adjusting the drug dosage at clinical work.

The clinical studies on Fengshiping were carried on in China, Japan and Austrilia. Theses studies were operated according to international criterion related disease classification about diagnosis, therapy and curative effect. By using the Fengshiping capsules Sololy, its effective rate was around 94%, and its remarkable effective rate was around 60%. It could improve the symptoms such as morning stiffness, swelling and pain and so on and the related items. The results showed in table 14˜21.

TABLE 14
Compared effect of treatment group with control group

| | | remission | No- | | | Notable | Effective |
|---|---|---|---|---|---|---|---|
| | | (clinical | table | Effec- | No | effect | rate |
| Groups | Cases | recovery) | effect | tive | effect | rate (%) | (%) |
| Treat- | 32 | 5 | 14 | 11 | 2 | 59.38 | 93.74 |
| ment | | | | | | | |
| group | | | | | | | |
| Control | 30 | 3 | 10 | 12 | 5 | 43.33 | 83.33 |
| group | | | | | | | |

TABLE 15
Influence of IgG, IgA and IgM (X ± S)

| | | IgG | | IgA | | IgM | |
|---|---|---|---|---|---|---|---|
| Groups | cases | pre- | post- | pre- | post- | pre- | post- |
| Normal | 32 | 12.45 ± 1.48 | | 2.37 ± 1.00 | | 1.58 ± 0.59 | |
| Treatment | 32 | 16.92 ± 3.49 | 14.17 ± 1.39** | 3.65 ± 1.03 | 2.39 ± 1.18** | 1.89 ± 0.88 | 1.48 ± 1.01 |
| group | | | | | | | |
| Control | 30 | 17.03 ± 4.12 | 15.14 ± 2.21** | 3.45 ± 1.86 | 2.32 ± 1.75** | 2.03 ± 0.95 | 1.76 ± 1.28 |

Comparing with pre-treatment **P < 0.01

TABLE 16
Influence of C3 and C4(X ± S)

| | | C3 | | C4 | |
|---|---|---|---|---|---|
| groups | cases | pre- | post- | pre- | post- |
| normal group | 32 | 0.62 ± 0.13 | | 0.14 ± 0.15 | |
| Treatment group | 32 | 1.88 ± 0.72 | 1.25 ± 0.66** | 0.48 ± 0.12 | 0.26 ± 0.06* |
| Control group | 30 | 2.13 ± 0.64 | 1.56 ± 0.62** | 0.40 ± 0.16 | 0.25 ± 0.07** |

Comparing with before therapy *P < 0.05, **P < 0.01

TABLE 17
Influence of ESR and CRP (X ± S)

| | | ESR | | CRP | |
|---|---|---|---|---|---|
| Groups | cases | pre- | post- | pre- | post- |
| Normal | 32 | 8.37 ± 5.26 | | 4.12 ± 1.88 | |
| Treatment | 32 | 66.58 ± 9.01 | 30.31 ± 6.53** | 13.35 ± 6.67 | 8.86 ± 3.34* |
| control | 30 | 73.33 ± 9.09 | 35.83 ± 11.61** | 14.21 ± 6.29 | 9.04 ± 3.15** |

Comparing with pre-treatment *P < 0.05, **P < 0.01

TABLE 18
Compared with power of gripping pre- and
post-treatment (X ± S)

| | Treatment Group | | Control Group | |
|---|---|---|---|---|
| groups | pre- | post- | pre- | post- |
| Gripping power of left | 39.13 ± 20.24(15) | 80.47 ± 34.61**(15) | 24.00 ± 17.63(21) | 55.15 ± 23.27**(21) |
| hands (mmHg) | | | | |
| Right hands | 35.85 ± 22.46(15) | 85.32 ± 36.32**(15) | 22.80 ± 12.32(21) | 58.17 ± 20.59**(21) |

Comparing with pre-treatment *P < 0.05, **P < 0.01

TABLE 19
Influence of arthrosis swelling and pain and morning
stiffness time (X ± S)

| | |
|---|---|
| Treatment Group | Control Group |

| Items | pre- | post- | pre- | post- |
|---|---|---|---|---|
| arthrosis swelling and pain | 5.79 ± 0.52 | 3.14 ± 0.83* | 5.56 ± 2.15 | 3.92 ± 0.26* |
| morning stiffness time | 50.33 ± 6.47 | 20.24 ± 3.27** | 48.75 ± 8.34 | 27.50 ± 3.78** |
| (minute) | | | | |

Comparing with pre-treatment *P < 0.05, **P < 0.01

TABLE 20
Influence of RF changing to negative

| | | RF negative | | |
|---|---|---|---|---|
| | | Pre- | Post- | Rate of negative |
| Groups | Cases | treatment | treatment | turnaround (%) |
| Treatment | 32 | 24 | 11 | 54.2 |
| group | | | | |
| Control | 30 | 18 | 10 | 44.4 |
| group | | | | |

Not only had significant effects, but also Fengshiping can make items such as SIL-2R, STNF, SIL-6R in plasma decrease, results showing in the Table 21.

TABLE 21
inflence of main indes such as SIL-2R, STNF α nd
SIL-6R (X ± S)

| | | SIL-2R(u/ml) | | STNF R1(ng/ml) | | SIL-6R(ng/ml) | |
|---|---|---|---|---|---|---|---|
| groups | Cases | pre- | post- | pre- | post- | pre- | post- |
| Normal | 32 | 299 ± 68 | | 1.56 ± 0.48 | | 72.05 ± 18.26 | |
| | | (n = 32) | | (n = 24) | | (n = 22) | |
| Fengshiping | 15 | 683 ± 189 | 381 ± 157** | 2.87 ± 0.66 | 1.75 ± 0.54** | 136.18 ± 28.57 | 90.15 ± 20.12** |
| Control | 10 | 765 ± 203 | 412 ± 167** | 2.63 ± 0.72 | 2.38 ± 0.39 | 148.21 ± 30.31 | 99.02 ± 26.70** |

(n = 8)

Comparing with pre-treatment **P < 0.01

It was proved that the above-mentioned results on invention could be realized on the ways as following.

### PRACTICE EXAMPLE 1

Epimedium brevicornum Maxim. 2222 g
Tripterygium hypoglaucum (Levl.) Hutch. 2222 g
Lycium barbarum L. 1111 g
Cuscuta chinensis Lam. 1111 g

Four herbs hereinbefore, Tripterygium hypoglaucum (Levl.) Hutch. was cut into pieces, extracted for three times after 13, 10, 10-fold added in, each time lasting 1 hour; Epimedium brevicornum Maxim was cut into segments, extracted three times after 15, 10, 10-fold water was added in, each extraction lasting 1 hour; Lycium barbarum L. was crushed to raw material, and immersed in 20-fold water of 80° C. for 1 hour; Cuscuta chinensis Lam. was crushed to raw powder, immersed in 31-fold water of 80° C. for 1 hour; decoction fluid or immersion fluid of four herbs were filtrated repectively, poured across macropore polymeric adsorbent column, eluted with 70% ethanol. When the color of effluent became deep significantly, eluent was commenced to collect; when the color of effluent became very weak, elution collection was ended. Eluent of each herb was recycled to get ethanol. Then the fluid without alcohol was concentrated, dried to get the finally extractive drug powder; officinal starch was blended with the four kinds of drug powder to 200 g, mixed up uniformly and encapsuled into 1000 capsules. Each capsule which was prepared with the invented method thereof, was composed of 0.2 g drugs extractive and contained at least 2.0 mg of icariine C₃₃H₄₀O₁₅. The regular dosage is: oral administration, three times every day, three capsules every time.

### PRACTICE EXAMPLE 2

Tripterygium hypoglaucum (Levl.) Hutch. 2000 g
Epimedium brevicornum Maxim. 2000 g

Two herbs hereinbefore, Tripterygium hypoglaucum (Levl.) Hutch. were cut into pieces, extracted three times after 13, 10, 10-fold added in, each time lasting 1 hour; Epimedium brevicornum Maxim. was cut into segments, extracted three times after 15, 10, 10-fold water was added in, each extraction lasting 1 hour; decoction fluid of herbs were filtrated repectively, poured across macropore polymeric adsorbent column, eluted with 70% ethanol, when the color of effluent became deep significantly, eluent was commenced to collect; when the color of effluent became very weak, elution was over. Eluent of each herbs was recycled to get ethanol, concentrated, dried, finally extractive drug powder was obtained; officinal starch was blended with the extractive drug powder, and mixed up uniformly, loaded to 1000 capsules. Each capsule which was prepared with the inventive method thereof, is composed of 0.2 g drugs extractive, contains at least 2.0 mg of icariine C33H40O15. regular dosage is: oral administration, three times every day, three capsules every time.

### PRACTICE EXAMPLE 3

Tripterygium hypoglaucum (Levl.) Hutch. 2000 g
Epimedium brevicornum Maxim. 2000 g
Lycium barbarum L. 1000 g

Tripterygium hypoglaucum (Levl.) Hutch. were cut into pieces, extracted three times after 13, 10, 10-fold added in, each time lasting 1 hour; Epimedium brevicornum Maxim. was cut into segments, extracted three times after 15, 10, 10-fold water was added in, each extraction lasting 1 hour; Lycium barbarum L. was crushed to raw material, and immersed in 20-fold water of 80° C. for 1 hour; decoction fluid or immersion fluid of four herbs were filtrated repectively, poured across macropore polymeric adsorbent column, eluted with 70% ethanol, when the color of effluent became deep significantly, eluent was commenced to collect; when the color of effluent became very weak, elution was over. Eluent of each herbs was recycled to get ethanol, concentrated, dried, finally extractive drug powder was obtained; officinal starch was blended with the extractive drug powder, and mixed up uniformly, loaded to 1000 capsules. Each capsule which was prepared with the inventive method thereof, is composed of 0.2 g drugs extractive, contains at least 2.0 mg of icariine C₃₃H₄₀O₁₅. Regular dosage is: oral administration, three times every day, three capsules every time.

### PRACTICE EXAMPLE 4

Tripterygium hypoglaucum (Levl.) Hutch. 2000 g
Epimedium brevicornum Maxim. 2000 g
Cuscuta chinensis Lam. 1000 g

Tripterygium hypoglaucum (Levl.) Hutch. were cut into pieces, extracted three times after 13, 10, 10-fold added in, each time lasting 1 hour; Epimedium brevicornum Maxim. was cut into segments, extracted three times after 15, 10, 10-fold water was added in, each extraction lasting 1 hour; Cuscuta chinensis Lam. was crushed to raw powder, immersed in 31-fold water of 80° C. for 1 hour; decoction fluid or immersion fluid of the herbs were filtrated repectively, poured across macropore polymeric adsorbent column, eluted with 70% ethanol, when the color of effluent became deep significantly, eluent was commenced to collect; when the color of effluent became very weak, elution was over. Eluent of each herbs was recycled to get ethanol, concentrated, dried, finally extractive drug powder was obtained; officinal starch was blended with extractive drug powder, and mixed up uniformly, loaded to 1000 capsules. Each capsule which was prepared with the inventive method thereof, is composed of 0.2 g drugs extractive, contains at least 2.0 mg of icariine C₃₃H40O15. Regular dosage is: oral administration, three times every day, three capsules every time.

### PRACTICE EXAMPLE 5

Tripterygium hypoglaucum (Levl.) Hutch. 2000 g
Cuscuta chinensis Lam. 1000 g

Tripterygium hypoglaucum (Levl.) Hutch. Were cut into pieces, extracted three times after 13, 10, 10-fold added in, each time lasting 1 hour; Cuscuta chinensis Lam. was crushed to raw powder, immersed in 31-fold water of 80° C. for 1 hour; decoction fluid or immersion fluid of the herbs were filtrated repectively, poured across macropore polymeric adsorbent column, eluted with 70% ethanol, when the color of effluent became deep significantly, eluent was commenced to collect; when the color of effluent became very weak, elution was over. Eluent of each herbs was recycled to get ethanol, concentrated, dried, finally extractive drug powder was obtained; officinal starch was blended with extractive drug powder, and mixed up uniformly, loaded to 1000 capsules. Dose of capsules administered every day, which was prepared with the inventive method thereof, was equivalent to dose of 30 g/day of crude drugs.

### PRACTICE EXAMPLE 6

Tripterygium hypoglaucum (Levl.) Hutch. 2000 g
Lycium barbarum L. 1000 g

Tripterygium hypoglaucum (Levl.) Hutch. were cut into pieces, extracted three times after 13, 10, 10-fold added in, each time lasting 1 hour; Lycium barbarum L. was crushed to raw material, and immersed in 20-fold water of 80° C. for 1 hour; decoction fluid or immersion fluid of the herbs were filtrated repectively, poured across macropore polymeric adsorbent column, eluted with 70% ethanol, when the color of effluent became deep significantly, eluent was commenced to collect; when the color of effluent became very weak, elution was over. Eluent of each herbs was recycled to get ethanol, concentrated, dried, finally extractive drug powder was obtained; officinal starch was blended with extractive drug powder, and mixed up uniformly, loaded to 1000 capsules. Dose of capsules administered every day, which was prepared with the inventive method thereof, was equivalent to dose of 30 g/day of crude drugs.

1. A pharmaceutical composition for treating rheumatism, characterized in that, it is made from the following materials:
Tripterygium hypoglaucum (Levl.) Hutch.
Epimedium brevicornum Maxim.
Lycium barbarum L.
Cuscuta chinensis Lam., Cuscuta australis R. Br.
Wherein the materials must be composed of Tripterygium hypoglaucum (Levl.) Hutch and one or two or three other herbs in the rest 3 herbs.

2. The pharmaceutical composition according to claim 1 made from the following materials:
Tripterygium hypoglaucum (Levl.) Hutch. 1-4 part by weight
Epimedium brevicornum Maxim. 1-4 part by weight
Lycium barbarum L. 1-4 part by weight
Cuscuta chinensis Lam., Cuscuta australis R. Br. 1-4 part by weight.

3. The pharmaceutical composition according to claim 1 made from the following materials:
Tripterygium hypoglaucum (Levl.) Hutch. 2 part by weight
Epimedium brevicornum Maxim. 2 part by weight
Lycium barbarum L. 1 part by weight
Cuscuta chinensis Lam., Cuscuta australis R. Br. 1 part by weight.

4. The pharmaceutical composition according to claim 1, characterized in that, it can be made from the correspond effective constituents of the materials above-mentioned as following that Epimedium brevicornum Maxim. can be replaced by any one or more than one among icariine, deuteron-icariine I, deuteron-icariine II and glyc-icariine A;
Tripterygium hypoglaucum (Levl.) Hutch can be replaced by diterpenoids, triterpenoids and alkaloids compound thereof, and Lycium barbarum L. and Cuscuta chinensis Lam. can be replaed by flavone contained thereof.

5. A method of preparing the pharmaceutical composition according to claim 1, 2 or 3, characterized in that, it includes the processes under-mentioned:
The raw herbs are weighed, and Epimedium brevicornum Maxim. and Tripterygium hypoglaucum (Levl.) Hutch. were cut into pieces respectively; including raw material or crushed powder of Lycium barbarum L. and Cuscuta chinensis Lam., four herbs hereinbefore, were extracted with 0-95% ethanol at 10-98° C. respectively or combinatively for continuing 1-4 times. Ethanol was recycled respectively or combinatively in extracted fluid, then extraction was concentrated, dried, crushed, mixed uniformly or proportionally, manufactured to dosage form adopted in clinical work;
Raw herbs were weighed: Epimedium brevicornum Maxim. and Tripterygium hypoglaucum (Levl.) Hutch. were cut into pieces, boiled out in water for three times respectively, and Lycium barbarum L. or Cuscuta chinensis Lam. were immersed in water of 80° C.˜95° C. for 1-3 times respectively. Decoction or immersion fluids of three times of each herb were blended respectively, then mixture fluid was respectively poured through corresponding macropore polymeric adsorbent column. After absorption, resin column was washed with water until effluent became clear, then was eluted with 30-99.5% ethanol until color of effluent became deep. Then eluent was collected until color of eluent became from deep to very weak while ethanol liquid was forced out from the column with water. Eluent was mixed with the ethanol liquid. The weight of total eluent was 1-8 fold of the herbs; eluent of each herbs was recycled, concentrated to specific gavity of 1.10 respectively, then extractive of every herbs were obtained by respective or combinative spray drying, which were mixed uniformly and proportionally, manufactured to dosage form adopted in clinical work.

6. A method of preparing the pharmaceutical composition according to claim 1, 2 or 3, characterized in that, it can be made into any dose forms adopted in the clinical work such as hard gelatin capsule, soft capsule, tablet, granule and injection.

7. A method of preparing the pharmaceutical composition according to claim 1, 2 or 3, characterized in that, it includes the processes under-mentioned:
Tripterygium hypoglaucum (Levl.) Hutch. were cut into pieces, extracted three times after 13, 10, 10-fold added in respectively, each time lasting 1 hour; Epimedium brevicornum Maxim. was cut into segments, extracted three times after 15, 10, 10-fold water was added in respectively, each extraction lasting 1 hour; Lycium barbarum L. was crushed to raw material, and immersed in 20-fold water of 80° C.-95° C. for 1 hour; Cuscuta chinensis Lam. was crushed to raw powder, immersed in 31-fold water of 90° C. for 1 hour; decoction fluid or immersion fluid of four herbs were filtrated repectively, poured through WLD or D101 or other type of macropore polymeric adsorbent column, eluted with 70% ethanol, when the color of effluent became deep significantly, eluent was commenced to collect; when the color of effluent became very weak, elution was over. Eluent of each herbs was recycled to get ethanol, concentrated, dried, finally extractive drug powder was obtained; which were mixed uniformly and proportionally, manufactured to dosage form adopted in clinical work.

8. The use of the pharmaceutical composition according to claim 1, 2 or 3 in the manufacture of a medicament for treating the rheumatoid and rheumatoid arthritis.

9. The use of the pharmaceutical composition according to claim 1, 2 or 3 in the manufacture of a medicament for treating the systemic lupus erythematosus.

10. The use of the pharmaceutical composition according to claim 1, 2 or 3 in the manufacture of a medicament for treating the chronic nephritis, crohn's disease and lepra reaction and the other autoimmune disease.
